(19) United States
(12) Patent Application Publication
(10) Pub. No.: US 2026/0079170 A1
(43) Pub. Date: Mar. 19, 2026

(54) METHODS FOR TREATING AND MONITORING PROGRANULIN-ASSOCIATED DISORDERS

(71) Applicant: Denali Therapeutics Inc., South San Francisco, CA (US)

(72) Inventors: Giuseppe Astarita, South San Francisco, CA (US); Sarah L. Devos, South San Francisco, CA (US); Gilbert Di Paolo, South San Francisco, CA (US); Todd P. Logan, South San Francisco, CA (US); Junhua Wang, South San Francisco, CA (US)

(73) Assignee: Denali Therapeutics Inc., South San Francisco, CA (US)

(21) Appl. No.: 19/334,563

(22) Filed: Sep. 19, 2025

Related U.S. Application Data
(63) Continuation of application No. 17/284,764, now abandoned.
(60) Provisional application No. 62/746,293, filed on Oct. 16, 2018.
(60) Provisional application No. 62/884,492, filed on Aug. 8, 2019.

(51) Int. Cl. G01N 33/92

(52) U.S. Cl. CPC G01N 33/92; G01N 2560/00; G01N 2800/2814; G01N 2800/50; G01N 2800/52

(57) ABSTRACT

The present disclosure provides methods and materials for screening a compound or monitoring a subject's response to a compound or dosing regimen for treating a PGRN-associated disorder. Methods and materials for identifying and treating a subject having a PGRN-associated disorder are also provided.

### CROSS-REFERENCE TO RELATED APPLICATIONS

This application is a continuation of U.S. patent application Ser. No. 17/284,764, filed Apr. 12, 2021, which is a national stage entry of International Application No. PCT/US2019/056351, filed Oct. 15, 2019, which claims priority to U.S. Provisional Application No. 62/746,293, filed Oct. 16, 2018, and U.S. Provisional Application No. 62/884,492, filed Aug. 8, 2019, the disclosures of which are hereby incorporated by reference in their entirety for all purposes.

### REFERENCE TO A SEQUENCE LISTING

The Sequence Listing written in file 638967SEQLST.xml is 3,649 bytes, was created on Sep. 19, 2025 and is hereby incorporated by reference.

### BACKGROUND

Frontotemporal dementia (FTD) is a progressive neurodegenerative disorder which accounts for 5-10% of all patients with dementia and 10-20% of patients with an onset of dementia before 65 years (Rademakers et al., Nat Rev Neurol. 8(8):423-34, 2012). While several genes have been linked to FTD, one of the most frequently mutated genes in FTD is GRN, which maps to human chromosome 17q21 and encodes the cysteine-rich protein progranulin (also known as proepithelin and acrogranin). Highly-penetrant mutations in GRN were first reported in 2006 as a cause of autosomal dominant forms of familial FTD (Baker et al., Nature. 442(7105):916-9, 2006; Cruts et al., Nature. 2006 Aug. 24; 442(7105):920-4; Gass et al., Hum. Mol. Genet. 15(20):2988-3001, 2006). Recent estimates suggest that GRN mutations account for 5-20% of FTD patients with positive family history and 1-5% of sporadic cases (Rademakers et al., supra). There is a need for new methods for evaluating compounds and related therapies for treating PGRN-associated disorders, including monitoring patient responses to such treatment, as well as for identifying subjects who can benefit from treatment of these disorders

### DESCRIPTION

In one aspect, the present disclosure provides a method for evaluating a compound or monitoring a subject's response to a compound, pharmaceutical composition, or dosing regimen thereof for treating a progranulin (PGRN)-associated disorder, the method comprising: (a) measuring an abundance of one or more bis(monoacylglycero)phosphate (BMP) species in a test sample from a subject having a PGRN-associated disorder, wherein the test sample or subject has been treated with the compound or pharmaceutical composition thereof; (b) comparing the difference in abundance between the one or more BMP species measured in (a) and one or more reference values; and (c) determining from the comparison whether the compound, pharmaceutical composition, or dosing regimen thereof improves one or more BMP species levels for treating a PGRN-associated disorder.

In some embodiments, the methods provided herein further comprise treating another test sample or subject with another compound and selecting a candidate compound that improves the one or more BMP species levels.

In some embodiments, the methods provided herein further comprise (d) maintaining or adjusting the amount or frequency of administration of the compound to the test sample or subject; and (e) administering the compound to the test sample or to the subject.

In one aspect, provided is a method for identifying a subject having, or at risk of having, a PGRN-associated disorder, the method comprising: (a) measuring the abundance of one or more BMP species in a test sample from the subject; (b) comparing the difference in abundance between the one or more BMP species measured in (a) and one or more reference values; and (c) determining from the comparison whether the subject has a PGRN-associated disorder.

In some embodiments, the methods provided herein further comprise administering to the subject a compound for improving the one or more BMP species levels for treating a PGRN-associated disorder. In some embodiments, at least one of the one or more signs or symptoms of a PGRN-associated disorder are ameliorated following treatment.

In some embodiments, treatment comprises administering PGRN, a derivative thereof, or a pharmaceutical composition thereof to the subject. In some embodiments the PGRN derivative contains a chemical moiety or peptide fragment that allows the PGRN to cross the blood brain barrier. In some embodiments, treatment comprises administering a library of compounds to a plurality of subjects or test samples.

In some embodiments, the reference value is measured in a reference sample obtained from a reference subject or a population of reference subjects. In some embodiments, the reference value is the abundance of the one or more BMP species measured in a reference sample. In some embodiments, the reference sample is the same type of cell, tissue, or fluid as the test sample. In some embodiments, at least two reference values from different types of cell, tissue, or fluid is measured.

In some embodiments, the reference sample is a healthy control. In some embodiments, the reference subject or population of reference subjects do not have a PGRN-associated disorder or a decreased level of PGRN. In particular embodiments, the reference subject or population of reference subjects do not have any signs or symptoms of such a disorder.

In some embodiments, BMP species levels are increased in bone marrow-derived macrophages that are derived in vitro from bone marrow cells of a subject having, or at risk of having, a PGRN-associated disorder as compared to a healthy control or a control not related to a PGRN-associated disorder.

In some embodiments, BMP species levels are decreased in liver, brain, cerebrospinal fluid, plasma, or urine of a subject having, or at risk of having, a PGRN-associated disorder as compared to a healthy control or a control not related to a PGRN-associated disorder.

In some embodiments, the abundance of a BMP species in the test sample of a subject having, or at risk of having, a PGRN-associated disorder has at least about a 1.2-fold, 1.5-fold, or 2-fold difference compared to a reference value of a control such as a healthy control or a control not related to a PGRN-associated disorder. In other embodiments, the abundance of a BMP species in the test sample of a subject having, or at risk of having, a PGRN-associated disorder has about a 1.2-fold to about 4-fold difference compared to a reference value of a control such as a healthy control or a control not related to a PGRN-associated disorder. In some embodiments, the difference compared to a reference value is about 2-fold to about 3-fold. In some embodiments, the subject has a disorder associated with a decreased level of PGRN and/or one or more signs or symptoms of a disorder associated with a decreased level of PGRN.

In some embodiments, the reference value is the BMP species value prior to treatment. In some embodiments, the subject is treated for a decreased level of PGRN or a PGRN-associated disease, and the test sample comprises one or more pre-treatment test samples that are obtained from the subject before treatment has started and one or more post-treatment test samples that are obtained from the subject after treatment has started. In some embodiments, the method further comprises determining that the subject is responding to the treatment when the abundance of at least one of the one or more BMP species post-treatment shows an improvement over the one or more BMP species pre-treatment relative to a healthy control.

In some embodiments, the methods comprise (a) measuring an abundance of one or more bis(monoacylglycero)phosphate (BMP) species in a test sample obtained from a subject; (b) treating the test sample or subject with a compound, pharmaceutical composition, or dosing regimen thereof; (c) measuring an abundance of one or more BMP species in a test sample obtained from the treated subject, and (d) comparing the abundance of the one or more BMP species measured in steps (a) and (c); and (c) determining whether the compound or a dosing regimen improves BMP levels for treating a PGRN-associated disorder.

In some embodiments, two or more post-treatment test samples are obtained at different time points after treatment has started, and the method further comprises determining that the subject is responding to treatment when the abundance of at least one of the one or more BMP species measured in a post-treatment sample is a) lower in bone marrow-derived macrophage (BMDM) or b) higher in liver, brain, cerebrospinal fluid, plasma, or urine than the abundance of the corresponding one or more BMP species measured in the pre-treatment sample. In some embodiments, the subject is determined to be responding to the treatment when the abundance of at least one of the one or more BMP species measured in a post-treatment sample is a) at least about 1.2-fold lower in BMDM or b) at least about 1.2-fold higher in liver, brain, cerebrospinal fluid, plasma, or urine than the abundance of the corresponding one or more BMP species measured in the pre-treatment sample.

In some embodiments, the improved BMP species level is an improvement over the BMP species level prior to treatment relative to the reference value of a control such as a healthy control or a control not related to a PGRN-associated disorder. In some embodiments, the improved BMP species level is closer in value to the control than the pre-treatment BMP species level is to the control. In some embodiments, the improved BMP species level has a difference compared to the control of less than 15%, 10%, or 5%. In some embodiments, the improved BMP species level has a difference compared to a healthy control of less than 10% or 5%. In some embodiments, the improved BMP species level has a difference compared to a healthy control of less than 5%.

In some embodiments, the method further comprises determining that the subject is responding to the treatment when the abundance of at least one of the one or more BMP species measured in at least one of the one or more post-treatment test samples is about the same as the corresponding reference value of a healthy control.

In some embodiments, the test or reference sample or one or more reference values comprises or relates to a cell, a tissue, whole blood, plasma, serum, cerebrospinal fluid, interstitial fluid, sputum, urine, feces, bronchioalveolar lavage fluid, lymph, semen, breast milk, amniotic fluid, or a combination thereof. In some embodiments, the cell is a peripheral blood mononuclear cell (PBMC), a bone marrow-derived macrophage (BMDM), a retinal pigmented epithelial (RPE) cell, a blood cell, an erythrocyte, a leukocyte, a neural cell, a microglial cell, a brain cell, a cerebral cortex cell, a spinal cord cell, a bone marrow cell, a liver cell, a kidney cell, a splenic cell, a lung cell, an eye cell, a chorionic villus cell, a muscle cell, a skin cell, a fibroblast, a heart cell, a lymph node cell, or a combination thereof. In some embodiments, the cell is a cultured cell. In some embodiments, the cultured cell is a BMDM or an RPE cell.

In some embodiments, the tissue comprises brain tissue, cerebral cortex tissue, spinal cord tissue, liver tissue, kidney tissue, muscle tissue, heart tissue, eye tissue, retinal tissue, a lymph node, bone marrow, skin tissue, blood vessel tissue, lung tissue, spleen tissue, valvular tissue, or a combination thereof. In some embodiments, the test and/or reference sample is purified from a cell and/or a tissue and comprises an endosome, a lysosome, an extracellular vesicle, an exosome, a microvesicle, or a combination thereof.

In some embodiments, the one or more BMP species comprise two or more BMP species. In some embodiments, the one or more BMP species comprise BMP(16:0_18:1), BMP(16:0_18:2), BMP(18:0_18:0), BMP(18:0_18:1), BMP(18:1_18:1), BMP(16:0_20:3), BMP(18:1_20:2), BMP(18:0_20:4), BMP(16:0_22:5), BMP(20:4_20:4), BMP(22:6_22:6), BMP(20:4_20:5), BMP(18:2_18:2), BMP(16:0_20:4), BMP(18:0_18:2), BMP(18:0e_22:6), BMP(18:1c_20:4), BMP(18:3_22:5), BMP(20:4_22:6), BMP(18:0e_20:4), BMP(18:2_20:4), BMP(18:1_22:6), BMP(18:1_20:4), BMP(18:0_22:6), or a combination thereof.

In some embodiments, the one or more BMP species comprise BMP(18:1_18:1), BMP(18:0_20:4), BMP(20:4_20:4), BMP(22:6_22:6), BMP(20:4_22:6), BMP(18:1_22:6), BMP(18:1_20:4), BMP(18:0_22:6), BMP(18:3_22:5), or a combination thereof.

In some embodiments, the test sample comprises a cultured cell and the one or more BMP species comprise BMP(18:1_18:1). In some embodiments, the test sample comprises plasma, tissue, urine, cerebrospinal fluid (CSF), and/or brain or liver tissue, and the one or more BMP species comprise BMP(22:6_22:6). In some embodiments, the test sample comprises liver tissue and the one or more BMP species comprise BMP(22:6_22:6), BMP(18:3_22:5), or a combination thereof. In some embodiments, the test sample comprises CSF or urine and the one or more BMP species comprise BMP(22:6_22:6). In some embodiments, the test sample comprises CSF and the one or more BMP species comprise BMP(18:1_18:1). In some embodiments, the test sample comprises microglia and the one or more BMP species comprise BMP(18:3_22:5).

In some embodiments, the abundance of the one or more BMP species is measured using a method selected from the group consisting of liquid chromatography-mass spectrometry (LC-MS), liquid chromatography-tandem mass spectrometry (LC-MS/MS), gas chromatography-mass spectrometry (GC-MS), gas chromatography-tandem mass spectrometry (GC-MS/MS), enzyme-linked immunosorbent assay (ELISA), and a combination thereof. In some embodiments, an internal BMP standard is used to measure the abundance of the one or more BMP species in step (a) and/or determine the corresponding reference value. In some embodiments, the internal BMP standard comprises a BMP species that is not naturally present in the subject and/or the reference subject or population of reference subjects. In some embodiments, the internal BMP standard comprises BMP(14:0_14:0).

In some embodiments, the PGRN-associated disorder is a disorder related to PGRN expression, processing, glycosylation, cellular uptake, trafficking, and/or function. In some embodiments, the subject and/or the reference subject or population of reference subjects have a decreased level of PGRN and/or a disorder associated with a decreased level of PGRN, and the test sample has been contacted with a candidate compound. In some embodiments, the subject and/or the reference subject or population of reference subjects have one or more signs or symptoms of the disorder associated with a decreased level of PGRN. In some embodiments, the subject and/or the reference subject or population of reference subjects have a mutation in a granulin (GRN) gene. In some embodiments, the mutation in the GRN gene decreases PGRN expression and/or activity. In some embodiments, the PGRN-associated disorder is atherosclerosis, Gaucher disease, or age-related macular degeneration (AMD). In some embodiments, the PGRN-associated disorder is Gaucher disease type 1. In some embodiments, the PGRN-associated disorder is a disorder associated with TDP-43. In other embodiments the TDP-43 associated disorder is AD or ALS.

In some embodiments, the PGRN-associated disorder is a neurodegenerative disease. In some embodiments, the neurodegenerative disease is selected from the group consisting of frontotemporal dementia (FTD), neuronal ceroid lipofuscinosis (NCL), Niemann-Pick disease type A (NPA), Niemann-Pick disease type B (NPB), Niemann-Pick disease type C (NPC), C9ORF72-associated amyotrophic lateral sclerosis (ALS)/FTD, sporadic ALS, Alzheimer's disease (AD), Gaucher disease types 2 and 3, and Parkinson's disease. In some embodiments, the subject and/or the reference subject is a human, a non-human primate, a rodent, a dog, or a pig.

In another aspect, the present disclosure provides a kit for monitoring PGRN levels in a subject. In some embodiments, the kit comprises a bis(monoacylglycero)phosphate (BMP) standard for measuring the abundance of one or more BMP species in a test sample obtained from the subject and/or a reference sample obtained from a reference subject or a population of reference subjects. In some embodiments, the BMP standard comprises a BMP species that is not naturally present in the subject and/or reference subject. In some embodiments, the BMP standard comprises BMP(14:0_14:0).

In some embodiments, the kit further comprises reagents for obtaining the sample from the subject and/or reference subject, processing the sample, measuring the abundance of the one or more BMP species, or a combination thereof. In some embodiments, the kit further comprises instructions for use.

### BRIEF DESCRIPTION OF THE DRAWINGS

FIGS. 1A and 1B show schematic drawings of fusion proteins F1 and F2, respectively, each containing a Fc polypeptide dimer and PGRN. In FIG. 1A, the N-terminus of PGRN is fused to the C-terminus of a Fc polypeptide dimer via a -GGGGSGGGGS- (SEQ ID NO:1) linker. In FIG. 1B, the C-terminus of PGRN is fused to the N-terminus of a Fc polypeptide dimer via a (G₄S)₂ (SEQ ID NO:1) linker. Fusion proteins F1 and F2 are further described in Example 1.

FIGS. 2A and 2B show increased levels of bis(monoacylglycero)phosphate (BMP) species in bone marrow-derived macrophages (BMDMs) of Grn knockout mice compared to wild-type. FIG. 2A shows that treating Grn knockout mouse BMDMs with PGRN fusion proteins F1 and F2 of Example 1 reduced elevated BMP(18:1_18:1) levels. FIG. 2B shows that treating Grn knockout mouse BMDMs with PGRN fusion proteins F1 and F2 of Example 1 reduced elevated BMP(20:4_20:4) levels.

FIGS. 2C and 2D show that treating Grn knockout mouse BMDMs with recombinant PGRN or PGRN expressed by lentivirus of Example 2 reduced elevated BMP levels (total BMP as well as BMP(18:1_18:1)). FIG. 2C shows data for total BMP. FIG. 2D shows data for BMP(18:1_18:1)

FIG. 3A shows decreased BMP 44:12 in the periphery liver, plasma, and urine of Grn knockout mice compared to wild-type. FIG. 3B shows decreased BMP 44:12 in cerebrospinal fluid (CSF) and brain of the central nervous system (CNS) compared to wild-type.

FIG. 4 shows that Grn knockout mice ranging from 2 to 19 months exhibited age-independent reduction in plasma BMP 44:12 compared to wild-type.

FIGS. 5A and 5B show that treatment of Grn knockout mice with PGRN fusion proteins F1 and F2 increased liver BMP 44:12 and 20:4_20:4 levels.

FIG. 6 shows that treatment of Grn knockout mice with PGRN fusion proteins F1 and F2 increased plasma BMP 44:12 levels.

FIG. 7 shows that treatment of Grn knockout mice with PGRN fusion proteins F1 and F2 increased urine BMP 44:12 levels (normalized to creatine).

FIG. 8 shows that treatment of Grn knockout mice with PGRN fusion proteins F1 and F2 increased CSF BMP 44:12 levels.

FIGS. 9A and 9B show that treatment of Grn knockout mice with PGRN fusion proteins F1 and F2 increased brain BMP 44:12 and 20:4_20:4 levels, respectively.

FIGS. 10A and 10B show decreased BMP 18:1_18:1 and 22:6_22:6 in sporadic and Grn FTD patient samples compared to clinically normal controls.

### DEFINITIONS

As used herein, the singular forms “a,” “an,” and “the” include plural referents unless the content clearly dictates otherwise. Thus, for example, reference to “a cell” may include two or more such cells, and the like.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

The term “abundance” refers to the amount or concentration of a molecule, compound, or agent (e.g., a bis(monoacylglycero)phosphate (BMP) molecule). The term includes an absolute amount or concentration, as well as a relative amount or concentration. In some embodiments, a reference standard (e.g., an internal BMP standard) is used for calibration in order to determine the absolute amount or concentration of a molecule, compound, or agent that is present (e.g., in a sample) and/or normalize to a control in order to determine a relative amount or concentration of a molecule, compound, or agent that is present.

The term “PGRN level” refers to the amount, concentration, and/or activity level of progranulin that is present, either in a subject or in a sample (e.g., a sample obtained from a subject). A PGRN level can refer to an absolute amount, concentration, and/or activity level of PGRN that is present, or can refer to a relative amount, concentration, and/or activity level. The term also refers to the amount or concentration of a PGRN polypeptide and/or PGRN mRNA (e.g., expressed from a GRN gene) that is present.

The term “progranulin” or “PGRN” (also known as “proepithelin” or “acrogranin”) refers to a cysteine-rich protein encoded by the gene GRN, which maps to human chromosome 17q21. PGRN is a lysosomal protein as well as a secreted protein consisting of seven and a half tandem repeats of conserved granulin peptides, each of which is about 60 amino acid long and can be released through cleavage by various extracellular proteases (e.g., elastase) and lysosomal proteases (e.g., cathepsin L) (Kao et al., Nat Rev Neurosci. 18(6):325-333, 2017). Generally, PGRN is believed to play both cell-autonomous and non-cell autonomous roles in the control of innate immunity as well as the function of lysosomes, where it regulates the activity and levels of various cathepsins and other hydrolases (Kao et al., supra). PGRN also has a neurotrophic function and promotes neurite outgrowth and neuronal survival (Kao et al., supra). A PGRN polypeptide may comprise a human PGRN sequence. A PGRN polypeptide may be a pre-mature PGRN, e.g., in which the first 17 amino acids make up the signal peptide. A PGRN polypeptide may also be a mature PGRN, e.g., in which the 17-amino acid signal peptide is cleaved. As other non-limiting examples, a PGRN polypeptide may comprise a sequence from a non-human species, such as mouse (NCBI reference number NP_032201.2), rat (NCBI reference number NP_058809.2 or NP_001139314.1), or chimpanzee (NCBI reference number XP_016787144.1 or XP_016787145.1) in either pre-mature or mature form. The term “progranulin derivative” or “PGRN derivative” refers to modified PGRN. The modifications can be made to increase the druggability of PGRN, such as modifications that target PGRN to specific areas in the body, and/or improve its pharmacokinetic or phamacodynamic properties. Other modifications can be made to assist in its manufacture and/or shelf life. PGRN derivatives can include Fc-fusion proteins comprising PGRN attached to dimers of Fc polypeptides.

The term “PGRN-associated disorder” refers to any pathological condition relating to PGRN including expression, processing, glycosylation, cellular uptake, trafficking, and/or function. The term “disorder associated with a decreased level of PGRN” refers to any pathological condition that directly or indirectly results from a level of PGRN that is insufficient to enable (i.e., is too low to enable) normal physiological function within a cell, a tissue, and/or a subject, as well as a precursors to such a condition. In some embodiments, the disorder is a neurodegenerative disease and/or a lysosomal storage disorder.

The term “bone marrow-derived macrophage” or “BMDM” refers to a macrophage cell that is generated or derived in vitro from a mammalian bone marrow (e.g., a bone marrow obtained from a subject). As a non-limiting example, BMDMs can be generated by culturing undifferentiated bone marrow cells in the presence of a cytokine such as macrophage colony-stimulating factor (M-CSF).

The terms “treatment,” “treating,” and the like are used herein to generally mean obtaining a desired pharmacologic and/or physiologic effect. “Treating” or “treatment” may refer to any indicia of success in the treatment or amelioration of a disease, including neurodegenerative diseases (e.g., frontotemporal dementia (FTD), atherosclerosis, Gaucher's disease, Alzheimer's disease (AD), neuronal ceroid lipofuscinosis (NCL), Niemann-Pick disease type A (NPA), Niemann-Pick disease type B (NPB), Niemann-Pick disease type C (NPC), C9ORF72-associated amyotrophic lateral sclerosis (ALS)/FTD, age-related macular degeneration (AMD), and Parkinson's disease), including any objective or subjective parameter such as abatement, remission, improvement in patient survival, increase in survival time or rate, diminishing of symptoms or making the disorder more tolerable to the patient, slowing in the rate of degeneration or decline, or improving a patient's physical or mental well-being. The treatment or amelioration of symptoms can be based on objective or subjective parameters. The effect of treatment can be compared to an individual or pool of individuals not receiving the treatment, or to the same patient prior to treatment or at a different time during treatment.

The term “subject,” “individual,” and “patient,” as used interchangeably herein, refer to a mammal, including but not limited to humans, non-human primates, rodents (e.g., rats, mice, and guinea pigs), rabbits, dogs, cows, pigs, horses, and other mammalian species. In one embodiment, the subject is a human.

As used herein, a “therapeutic amount” or “therapeutically effective amount” of an agent is an amount of the agent that treats symptoms of a disease in a subject.

The term “administer” refers to a method of delivering agents, compounds, or compositions to the desired site of biological action. These methods include, but are not limited to, topical delivery, parenteral delivery, intravenous delivery, intradermal delivery, intramuscular delivery, subcutaneous delivery, intrathecal delivery, oral delivery, colonic delivery, rectal delivery, or intraperitoneal delivery.

### Bis(monoacylglycero)phosphate (BMP)

Provided herein are methods of monitoring the levels of PGRN (e.g., in a sample, in a cell, in a tissue, and/or in a subject), wherein determining the level of PGRN comprises measuring the abundance of bis(monoacylglycero)phosphate (BMP) (e.g., in the sample, cell, tissue, and/or subject). BMP is a glycerophospholipid that is negatively charged (e.g. at the pH normally present within late endosomes and lysosomes) having the structure depicted in Formula I:

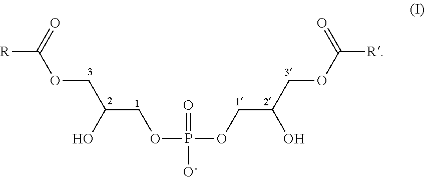

BMP molecules comprise two fatty acid side chains. R and R′ in Formula I represent independently selected saturated or unsaturated aliphatic chains, each of which typically contains 14, 16, 18, 20, or 22 carbon atoms. When a fatty acid side chain is unsaturated, it can contain 1, 2, 3, 4, 5, 6, or more carbon-carbon double bonds. Furthermore, a BMP molecule can contain one or two alkyl ether substituents, wherein the carbonyl oxygen of one or both fatty acid side chains is replaced with two hydrogen atoms.

Nomenclature that is used herein to describe a particular BMP species refers to a species having two fatty acid side-chains, wherein the structures of the fatty acid side chains are indicated within parentheses in the BMP format (e.g., BMP(18:1_18:1)). The numerals follow the standard fatty acid notation format of number of “fatty acid carbon atoms:number of double bonds.” An “e-” prefix is used to indicate the presence of an alkyl ether substituent wherein the carbonyl oxygen of the fatty acid side chain is replaced with two hydrogen atoms. For example, the “e” in “BMP(16:0e_18:0)” denotes that the side chain having 16 carbon atoms is an alkyl ether substituent.

BMP is unusual in that it has an sn-1;sn-1′ structural configuration (i.e., based on the phosphate-linked glycerol carbon) that is not observed in other glycerophospholipids. Synthesis of BMP involves a number of acylation and diacylation steps and involves transacylase activity, which reorients the glycerol backbone and produces the unusual structural configuration. The sn-1;sn-1′ configuration is believed to contribute to the resistance of BMP to cleavage by many phospholipases and its stability in late endosomes and lysosomes. While BMP is found in many different cell types in low amounts, BMP content is significantly higher in macrophages, as well as lysosomes in liver and other tissue types.

Consistent with their function as digestive organelles, lysosomes contain large amounts of hydrolytic enzymes at an acidic pH (i.e., a pH of about 4.6 to about 5). Various cellular constituents and foreign antigens are captured by receptors on the cell surface for uptake and delivery to lysosomes. Within the cell, receptors such as the mannose-6-phosphate receptor bind and divert hydrolytic enzymes from biosynthetic pathways to the lysosomes. The captured molecules pass through an intermediate heterogeneous set of organelles known as endosomes, which function as a sorting station where the receptors are recycled before hydrolases and other materials are directed to the lysosomes. There, the hydrolases are activated and the unwanted materials are digested. In particular, internal vesicles and/or membranes of mature or “late” endosomes and lysosomes contain large amounts of BMP.

Being negatively-charged at lysosomal pH, BMP can dock with luminal acid hydrolases that are positively charged at acidic pH and require a water-lipid interface for activation. By binding in this way, BMP can stimulate a number of lysosomal lipid-degrading enzymes, including acid sphingomyelinase, acid ceramidase, acid phospholipase A2, and an acid lipase that has the capacity to hydrolyze triacylglycerols and cholesterol esters.

Endosomal membranes are a continuation of lysosomal membranes, and they function to sort and recycle material back to the plasma membrane and endoplasmic reticulum. Accordingly, low-density lipoproteins (LDLs) that are internalized in cells reach late endosomes, where the constituent cholesterol esters and triglycerides are hydrolyzed by an acid lipase. The characteristic network of BMP-rich membranes contained within late endosomes or lysosomes is an important element of cholesterol homeostasis in that it regulates cholesterol transport by acting as a collection and re-distribution point for free cholesterol. For example, when lysosomal membranes are incubated with anti-BMP antibodies, substantial amounts of cholesterol accumulate.

In some embodiments of methods of the present disclosure, the abundance of a single BMP species is measured. In some embodiments, the abundance of two or more BMP species is measured. In some embodiments, the abundance of at least two, three, four, five, or more of the BMP species in Table 1 is measured. When the abundance of two or more BMP species is measured, any combination of different BMP species can be used.

In some embodiments, the abundance of more than one BMP species can be summed, and the total abundance will be compared to a reference value. For example, the abundance of each of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, or more BMP species (e.g., the BMP species listed in Table 1) can be summed, and the total abundance then compared to a reference value.

In some cases, one or more BMP species may be differentially expressed (e.g., more or less abundant) in one type of sample when compared to another, such as, for example, cell-based samples (e.g., cultured cells) versus tissue-based or blood samples. Accordingly, in some embodiments, the selection of the one or more BMP species (i.e., for the measurement of abundance) depends on the type of sample. In some embodiments, the one or more BMP species comprise BMP(18:1_18:1), e.g., when a sample (e.g., a test sample and/or a reference sample) is bone marrow-derived macrophage (BMDM). In other embodiments, the one or more BMP species comprise BMP(22:6_22:6), e.g., when a sample comprises tissue (e.g., brain tissue, liver tissue) or plasma, urine, or CSF.

In some embodiments, an internal BMP standard (e.g., BMP(14:0_14:0)) is used to measure the abundance of one or more BMP species in a sample and/or determine a reference value (e.g., measure the abundance of one or more BMP species in a reference sample). For example, a known amount of the internal BMP standard can be added to a sample (e.g., a test sample and/or a reference sample) to serve as a calibration point such that the amount of one or more BMP species that are present in the sample can be determined. In some embodiments, a reagent used in the extraction or isolation of BMP from a sample (e.g., methanol) is “spiked” with the internal BMP standard. Typically, the internal BMP standard will be one that does not naturally occur in the subject.

### Identification of Subjects Having, or at Risk of Having, a PGRN-Associated Disorder

In some embodiments, a subject (e.g., a target subject) is determined to have a PGRN-associated disease or a decreased level of PGRN when the abundance of at least one (e.g., a at least 1, 2, 3, 4, 5, 10, 15, 20, 25, 30, 35, 40, 45, 50, 55, 60, 65, 70, 75, 80, 85, 90, 95, 100, 105, 110, 115, 120, 125, 130, 135, 140, 145, or more) BMP species (e.g., the BMP species listed in Table 1) in a test sample is higher when the test sample is a BMDM or lower when the test sample is liver, brain, cerebrospinal fluid, plasma, or urine than a reference value of a corresponding cell, tissue, or fluid of a healthy control or a control not related to a PGRN-associated disorder.

In some embodiments, a subject (e.g., a target subject) is determined to have a PGRN-associated disease or a decreased level of PGRN when the abundance of at least one (e.g., 1, 2, 3, 4, 5, 6, 7, 8, 9, 10, 11, 12, 13, 14, 15, 16, 17, 18, 19, 20, 21, 22, or all 23) of the BMP species selected from the group consisting of BMP(16:0_18:1), BMP(16:0_18:2), BMP(18:0_18:0), BMP(18:0_18:1), BMP(18:1_18:1), BMP(16:0_20:3), BMP(18:1_20:2), BMP(18:0_20:4), BMP(16:0_22:5), BMP(20:4_20:4), BMP(22:6_22:6), BMP(20:4_20:5), BMP(18:2_18:2), BMP(16:0_20:4), BMP(18:0_18:2), BMP(18:0e_22:6), BMP(18:1c_20:4), BMP(20:4_22:6), BMP(18:0e_20:4), BMP(18:2_20:4), BMP(18:1_22:6), BMP(18:1_20:4), BMP(18:0_22:6), and BMP(18:3_22:5) is elevated in BMDM or decreased in liver, brain, cerebrospinal fluid, plasma, or urine compared to a reference value of a corresponding cell, tissue, or fluid of a healthy control or a control not related to a PGRN-associated disorder.

In some embodiments, a subject (e.g., a target subject) is determined to have a PGRN-associated disease or a decreased level of PGRN when the abundance of at least one (e.g., 1, 2, 3, 4, 5, 6, 7, or all 8) of the BMP species selected from the group consisting of BMP(18:1_18:1), BMP(18:0_20:4), BMP(20:4_20:4), BMP(22:6_22:6), BMP(20:4_22:6), BMP(18:1_22:6), BMP(18:1_20:4), BMP(18:0_22:6) and BMP(18:3_22:5) is elevated in BMDM or decreased in liver, brain, cerebrospinal fluid, plasma, or urine compared to a reference value of a corresponding cell, tissue, or fluid of a healthy control or a control not related to a PGRN-associated disorder.

In some embodiments, a subject is determined to have a PGRN-associated disease or a decreased level of PGRN when BMP(18:1_18:1) levels are elevated in BMDM compared to a reference value of a healthy control or a control not related to a PGRN-associated disorder. In other embodiments, a subject is determined to have a PGRN-associated disease or a decreased level of PGRN when BMP(22:6_22:6) are decreased in plasma, urine, cerebrospinal fluid (CSF), and/or brain or liver tissue compared to a reference value of a healthy control or a control not related to a PGRN-associated disorder. In other embodiments, a subject is determined to have a PGRN-associated disease or a decreased level of PGRN when BMP(22:6_22:6) and/or BMP(18:3_22:5) levels are decreased in liver tissue. In other embodiments, a subject is determined to have a PGRN-associated disease or a decreased level of PGRN when BMP(18:3_22:5) levels are decreased in microglia.

In some embodiments, a subject (e.g., a target subject) is determined to have a PGRN-associated disease or a decreased level of PGRN when the abundance of at least one of the BMP species (e.g., measured in a test sample) is at least about 1.1-fold (e.g., about 1.1-fold, 1.2-fold, 1.3-fold, 1.4-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, or more) higher in BMDM or lower in liver, brain, cerebrospinal fluid, plasma, or urine compared to a reference value of a corresponding cell, tissue, or fluid of a healthy control or a control not related to a PGRN-associated disorder.

In some embodiments, a subject (e.g., a target subject) is determined to have a PGRN-associated disease or a decreased level of PGRN when the abundance of at least one of the BMP species (e.g., measured in a test sample) is at least about 1.2-fold to about 4-fold (e.g., at least about 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, or 4-fold) higher than a reference value (e.g., a corresponding reference value). In some embodiments, a subject is determined to have a PGRN-associated disease or a decreased level of PGRN when the abundance of at least one of the BMP species is about 2-fold to about 3-fold (e.g., about 2-fold, 2.1-fold, 2.2-fold, 2.3-fold, 2.4-fold, 2.5-fold, 2.6-fold, 2.7-fold, 2.8-fold, 2.9-fold, or 3-fold) higher in BMDM or lower in liver, brain, cerebrospinal fluid, plasma, or urine compared to a reference value of a corresponding cell, tissue, or fluid of a healthy control or a control not related to a PGRN-associated disorder.

### Monitoring Response to Treatment

In one aspect, the present disclosure provides methods for monitoring PGRN levels in a subject (e.g., a target subject). In another aspects, provided are methods for monitoring a subject's response to a compound, pharmaceutical composition, or dosing regimen thereof or response to any therapy or therapeutic for treating a PGRN-associated disorder.

Typically, the abundance of each of the one or more BMP species in a test sample will be compared to one or more reference values (e.g., a corresponding reference value). In some embodiments, a BMP value is measured before treatment and at one or more time points after treatment. The abundance value taken at a later time point can be compared to the value prior to treatment as well as to a control value, such as that of a healthy or diseased control, to determine how the subject is responding to the therapy. The one or more reference values can be from different cells, tissues, or fluids corresponding to the cell, tissue, or fluid of the test sample.

In some embodiments, the reference value is the abundance of the one or more BMP species that is measured in a reference sample. The reference value can be a measured abundance value (e.g., abundance value measured in the reference sample), or can be derived or extrapolated from a measured abundance value. In some embodiments, the reference value is a range of values, e.g., when the reference values are obtained from a plurality of samples or a population of subjects. Furthermore, the reference value can be presented as a single value (e.g., a measured abundance value, a mean value, or a median value) or a range of values, with or without a standard deviation or standard of error.

When two or more test samples are obtained (e.g., from a subject), the time points at which they are obtained can be separated by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more minutes; about 1, 2, 3, 4, 5, 6, 7, 8, 9, 10, 11, 12, 13, 14, 15, 16, 17, 18, 19, 20, 21, 22, 23, 24, or more hours; about 1, 2, 3, 4, 5, 6, 7, or more days; about 1, 2, 3, 4, 5, 6, 7, 8, 9, 10, or more weeks; or even longer. When three or more test samples are obtained, the time intervals between when each test sample is obtained can all be the same, the intervals can all be different, or a combination thereof.

In some embodiments, both the first test sample and the second test sample are obtained from a subject (e.g., a target subject) after the subject has been treated, i.e., the first test sample is obtained from the subject at an earlier time point during treatment than the second test sample. In some embodiments, the first test sample is obtained before the subject has been treated for the disorder associated with a decreased level of PGRN (i.e., a pre-treatment test sample) and the second test sample is obtained after the subject has been treated for the disorder associated with a decreased level of PGRN (i.e., a post-treatment test sample). In some embodiments, more than one (e.g., 1, 2, 3, 4, 5, 6, 7, 8, 9, 10, or more) pre-treatment and/or post-treatment test samples are obtained from the subject. Furthermore, the number of pre-treatment and post-treatment test samples that are obtained need not be the same.

In some embodiments, it may be determined that the subject is not responding to the treatment when the abundance the BMP species measured is within about 1%, 2%, 3%, 4%, 5%, 6%, 7%, 8%, 9%, 10%, 11%, 12%, 13%, 14%, 15%, 16%, 17%, 18%, 19%, or 20% of the reference value.

When a subject (e.g., a target subject) is not responding to treatment (e.g., for a disorder associated with a decreased level of PGRN), in some embodiments, the dosage of one or more therapeutic agents (e.g., PGRN) is altered (e.g., increased) and/or the dosing interval is altered (e.g., the time between doses is decreased). In some embodiments, when a subject is not responding to treatment, a different therapeutic agent is selected. In some embodiments, when a subject is not responding to treatment, one or more therapeutic agents is discontinued.

### BMP Detection Techniques

In some embodiments, antibodies can be used to detect and/or measure the abundance of one or more BMP species. BMP species bound to the antibody can be detected such as by microscopy or enzyme-linked immunosorbent assay (ELISA).

In other embodiments, mass spectrometry (MS) is used to detect and/or measure the abundance of one or more BMP species according to methods of the present disclosure. Mass spectrometry is a technique in which compounds are ionized, and the resulting ions are sorted by their mass-to-charge ratios (abbreviated m/Q, m/q, m/Z, or m/z). A sample (e.g., comprising a BMP molecule), which can be present in gas, liquid, or solid form, is ionized, and the resulting ions are then accelerated through an electric and/or magnetic field, causing them to be separated by their mass-to-charge ratios. The ions ultimately strike an ion detector and a mass spectrogram is generated. The mass-to-charge ratios of the detected ions, together with their relative abundance, can be used to identify the parent compound(s), sometimes by correlating known masses (e.g., of entire or intact molecules) to the masses of the detected ions and/or by recognition of patterns that are detected in the mass spectrogram.

Mass spectrometers typically include at least four primary components: (1) a sample inlet device (e.g., a vaporizer), (2) an ionization device, (3) an ion path, and (4) an ion detector. In addition, mass spectrometers commonly comprise a device that converts samples into a form suitable for the inlet device and/or separates compounds that are present within the sample, which in some embodiments can be a chromatography device (e.g., liquid or gas chromatography) or a solid target for matrix-assisted laser desorption/ionization (MALDI) or another technique suitable for solid samples. Mass spectrometers also commonly comprise a device for signal processing of detector signals (e.g., an analog-digital converter (ADC)) and/or software for the processing, analysis, and display of detector signals.

The sample inlet device facilitates the transition of a solid or liquid specimen into the gaseous phase, which is required for subsequent processing and analysis. The ionization device can utilize, for example, hard ionization (e.g., electron ionization) or soft ionization (e.g., fast atom bombardment (FAB), chemical ionization (CI), electrospray ionization (ESI), MALDI, or atmospheric-pressure chemical ionization (APCI)). ESI methods comprise passing a solution through a length of capillary tube, to the end of which is applied a high positive or negative electric potential. Solution reaching the end of the tube is vaporized into a jet or spray comprising very small droplets of solution in solvent vapor. This spray of droplets flows through an evaporation chamber that is heated slightly to prevent condensation and to evaporate solvent. As the droplets get smaller, the electrical surface charge density increases until the natural repulsion between like charges causes ions as well as neutral molecules to be released.

In the ion path, ions transition from a near atmospheric pressure environment to the low pressure (e.g., high vacuum) environment of the mass analyzer, which separates the ions according to their mass-to-charge ratios, and are moved towards the ion detector. The ion detector is commonly an electron multiplier or a microchannel plate that releases a cascade of electrons in response to being struck by an ion.

Different types of mass analyzers can be used, examples of which include sector field mass analyzers, time-of-flight (TOF) mass analyzers, and quadrupole mass analyzers. Sector field mass analyzers use a static electric and/or magnetic field to modify the ion path and/or velocity, effectively bending the trajectories of the ions according to their mass-to-charge ratios. Ions with higher charges and/or lower masses will be deflected more than ions with lower charges and/or higher masses. A TOF mass analyzer uses an electric field to accelerate the ions through a specified potential, and the time that an ion takes to strike the detector is measured. If all of the ions have the same charge, then their velocities will only differ as a function of their masses, and ions with lower masses will strike the detector first. Quadrupole mass analyzers employ one or more sets of four parallel rods (a set of four parallel rods being known as a quadrupole) to generate oscillating electrical fields that stabilize or destabilize the paths of ions as they pass through an electric quadrupole field (e.g., radio frequency (RF) quadrupole file) that is created between the four rods. In particular, only ions within a specific range of mass-to-charge ratios are allowed to pass though the mass analyzer at any given time. However, by varying the potentials on the rods, a wide range of mass-to-charge ratios can be swept rapidly. Mass-to-charge ratios can be swept either continuously or by specifying discrete jumps.

As opposed to single mass spectrometry (MS) that uses a single mass analyzer (e.g., quadrupole), tandem mass spectrometry (MS/MS) uses a series of mass analyzers (e.g., three mass analyzers) to perform multiple rounds of mass spectrometry, typically having a molecule fragmentation step in between. As a non-limiting example, MS/MS instruments commonly employ three quadrupole mass analyzers. The first quadrupole (Q1) can act as a first mass filter, separating a species or protein of interest from a larger heterogeneous population. The second quadruple (Q2) can act as a collision chamber that stabilizes the ions that have passed through Q1 and can be filled with a low-pressure gas, with which the ions collide, causing them to fragment (collision-induced-fragmentation (CID)). The third quadrupole (Q3) can act as a second mass filter that separates the fragments produced in Q2 and passes them along to the detector. Alternatively, instead of performing tandem mass spectrometry in space, tandem mass spectrometry can be performed over time using a single mass analyzer, such as when a quadrupole ion trap is used and the field is varied over time. Briefly, a quadrupole ion trap works based on the same physical principles as a quadrupole mass analyzer, but the ions are trapped within the quadrupole (i.e., the electric field changes faster than the time required for the ions to escape) and are selectively ejected over time by varying the field generated by the quadrupole.

Several methods can be used for fragmentation, including but not limited to CID, electron capture dissociation (ECD), electron transfer dissociation (ETD), infrared multiphoton dissociation (IRMPD), blackbody infrared radiative dissociation (BIRD), electron-detachment dissociation (EDD), and surface-induced dissociation (SID).

Tandem mass spectrometers can be used to run different types of experiments, including full scans, product ion scans, precursor ion scans, neutral loss scans, and selective (or multiple) reaction monitoring (SRM or MRM) scans. In a full scan experiment, the entire mass range or a portion thereof) of both mass analyzers (e.g., Q1 and Q3) are scanned and the second mass analyzer (e.g., Q2) does not contain any collision gas. This allows all ions contained in a sample to be detected. In a product ion scan experiment, a specific mass-to-charge ratio is selected for the first mass analyzer (e.g., Q1), the second mass analyzer (e.g., Q2) is filled with a collision gas to fragment ions having the selected mass-to-charge ratio, and then the entire mass range (or a portion thereof) of the third mass analyzer (e.g., Q3) is scanned. This allows all fragment ions of a selected precursor ion to be detected. In a precursor ion scan experiment, the entire mass range (or a portion thereof) of the first mass analyzer (e.g., Q1) is scanned, the second mass analyzer (e.g., Q2) is filled with collision gas to fragment ions falling within the scan range, and a specific mass-to-charge ratio is selected for the third mass analyzer (e.g., Q3). By correlating the time between detection of a product ion and the particular mass-to-charge ratio that was selected just prior to its detection, this type of experiment can allow a user to determine which precursor ion(s) may have generated the product ion of interest. In a neutral loss scan experiment, the entire mass range (or a portion thereof) of the first mass analyzer (e.g., Q1) is scanned, the second mass analyzer (e.g., Q2) is filled with collision gas to fragment all ions within the scan range, and the third mass analyzer (e.g., Q3) is scanned across a specified range that corresponds to the fragmentation-induced loss of a single specific mass that has occurred for every potential ion in the precursor scan range. This type of experiment permits the identification of all precursors that have lost a particular chemical group of interest (e.g., a methyl group) in common. In an MRM experiment, one specific mass-to-charge ratio is selected for the first mass analyzer (e.g., Q1), the second mass analyzer (e.g., Q2) is filled with collision gas, and the third mass analyzer (e.g., Q3) is set for another specific mass-to-charge ratio. This type of experiment permits the highly specific detection of molecules that are known to fragment into the products that are selected for in the third mass analyzer. MS and MS/MS methods are described further in Grebe et al. Clin. Biochem. Rev. (2011) 32:5-31, hereby incorporated by reference in its entirety for all purposes.

Furthermore, MS and MS/MS techniques can be coupled with liquid chromatography (LC) or gas chromatography (GC) techniques. Such liquid chromatography-mass spectrometry (LC-MS), liquid chromatography-tandem mass spectrometry (LC-MS/MS), gas chromatography-mass spectrometry (GC-MS), and gas chromatography-tandem mass spectrometry (GC-MS/MS) methods allow for enhanced mass resolving and mass determining over what is typically possible with MS or MS/MS alone.

Liquid chromatography refers to a process in which one or more components of a fluid solution are selectively retarded as the fluid uniformly percolates through a column of a finely divided substance, or through capillary passageways. The retardation results from the distribution of the components of the mixture between one or more stationary phases and the bulk fluid (i.e., mobile phase), as the fluid moves relative to the stationary phase(s). High performance liquid chromatography (HPLC), also sometimes known as “high pressure liquid chromatography,” is a variant of LC in which the degree of separation is increased by forcing the mobile phase under pressure through a stationary phase, typically a densely packed column.

Furthermore, ultra high performance liquid chromatography (UHPLC), also known as “ultra high pressure liquid chromatography,” or “ultra performance liquid chromatography (UPLC),” is a variant of HPLC that is performed using much higher pressures than traditional HPLC techniques.

In some embodiments, the size of particles in the column is less than about 2.0 μm (e.g., about 1.9 μm, 1.8 μm, 1.7 μm, 1.6 μm, 1.5 μm, or smaller). In some embodiments, the particle size is about 1.7 μm.

In some embodiments, the working pressure on the column is about 400 bar to about 1,000 bar (e.g., about 400, 450, 500, 550, 600, 650, 700, 750, 800, 850, 900, 950, or 1,000 bar).

In some embodiments, during chromatography the temperature of the column is maintained at a temperature between about 40° C. and about 60° C. (e.g., about 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., or 60° C.). In some embodiments, the column is maintained at a temperature of about 55° C.

In some embodiments, the flow rate of the column is between about 0.10 mL/minute and about 0.50 mL/minute (e.g., about 0.10 mL/minute, 0.11 mL/minute, 0.12 mL/minute, 0.13 mL/minute, 0.14 mL/minute, 0.15 mL/minute, 0.16 mL/minute, 0.17 mL/minute, 0.18 mL/minute, 0.19 mL/minute, 0.20 mL/minute, 0.21 mL/minute, 0.22 mL/minute, 0.23 mL/minute, 0.24 mL/minute, 0.25 mL/minute, 0.26 mL/minute, 0.27 mL/minute, 0.28 mL/minute, 0.29 mL/minute, 0.30 mL/minute, 0.31 mL/minute, 0.32 mL/minute, 0.33 mL/minute, 0.34 mL/minute, 0.35 mL/minute, 0.36 mL/minute, 0.37 mL/minute, 0.38 mL/minute, 0.39 mL/minute, 0.40 mL/minute, 0.41 mL/minute, 0.42 mL/minute, 0.43 mL/minute, 0.44 mL/minute, 0.45 mL/minute, 0.46 mL/minute, 0.47 mL/minute, 0.48 mL/minute, 0.49 mL/minute, or 0.50 mL/minute). In some embodiments, the flow rate is about 0.40 mL/minute.

The gradient elution can be made using two solvents (e.g., A and B solvents). In some embodiments, the A solvent is 10 mM ammonium formate+0.1% formic acid in water and the B solvent is acetonitrile with 0.1% formic acid. In some embodiments, the gradient is produced by the following method: 5% A and 95% B for 1 minute, changed to 50% A and 50% B over 6 minutes, changed to 5% A and 95% B over 0.1 minutes, then maintained at 5% A and 95% B for 4.9 minutes. Once the compounds are separated by LC, HPLC, or UHPLC, they may be introduced into the mass spectrometer.

Gas chromatography refers to a method for separating and/or analyzing compounds that can be vaporized without being decomposed. The mobile phase is a carrier gas that is typically an inert gas (e.g., helium) or an unreactive gas (e.g., nitrogen), and the stationary phase is typically a microscopic liquid or polymer layer positioned on an inert solid support inside glass or metal tubing that serves as the “column.” As the gaseous compounds of interest interact with the stationary phase within the column, they are differentially retarded and eluted from the column at different times. The separated compounds can then be introduced into the mass spectrometer.

In some embodiments, antibody-based methods are used to detect and/or measure the abundance of one or more BMP species. Non-limiting examples of suitable methods include enzyme-linked immunosorbent assay (ELISA), immunofluorescence, and radioimmunoassay (RIA) techniques. Methods for performing ELISA, immunofluorescence, and RIA techniques are known in the art.

Any number of sample types can be used as a test sample and/or reference sample in methods of the present disclosure so long as the sample comprises BMP in an amount sufficient for detection such that the abundance can be measured. Non-limiting examples include cells, tissues, blood (e.g., whole blood, plasma, serum), fluids (e.g., cerebrospinal fluid, urine, bronchioalveolar lavage fluid, lymph, semen, breast milk, amniotic fluid), feces, sputum, or any combination thereof. Non-limiting examples of suitable cell types include bone marrow-derived macrophages (BMDMs), blood cells (e.g., peripheral blood mononuclear cells (PBMCs), erythrocytes, leukocytes), neural cells (e.g., brain cells, cerebral cortex cells, spinal cord cells), bone marrow cells, liver cells, kidney cells, splenic cells, lung cells, eye cells (e.g., retinal cells such as retinal pigmented epithelial (RPE) cells), chorionic villus cells, muscle cells, skin cells, fibroblasts, heart cells, lymph node cells, or a combination thereof. In some embodiments, the sample comprises a portion of a cell. In some embodiments, the sample is purified from a cell or a tissue. Non-limiting examples of purified samples include endosomes, lysosomes, extracellular vesicles (e.g., exosomes, microvesicles), and combinations thereof.

In some embodiments, the sample (e.g., test sample and/or reference sample) comprises a cell that is a cultured cell. Non-limiting examples include BMDMs and RPE cells. BMDMs can be obtained, for example, by procuring a sample comprising PBMCs and culturing the monocytes contained therein.

Non-limiting examples of suitable tissue sample types include neural tissue (e.g., brain tissue, cerebral cortex tissue, spinal cord tissue), liver tissue, kidney tissue, muscle tissue, heart tissue, eye tissue (e.g., retinal tissue), lymph nodes, bone marrow, skin tissue, blood vessel tissue, lung tissue, spleen tissue, valvular tissue, and a combination thereof. In some embodiments, a test sample and/or a reference sample comprises brain tissue or liver tissue. In some embodiments, a test and/or a reference sample comprises plasma.

Disorders Associated with PGRN Deficiency

PGRN is involved in the maintenance of lysosomal homeostasis and the regulation of lipid metabolism, and PGRN deficiency is associated with lysosomal dysfunction (Evers et al. Cell Reports 20:2565-2574, 2017). In turn, a number of diseases may be caused by or linked to lysosomal storage disorders characterized by the accumulation of undigested or partially digested macromolecules, which ultimately results in cellular and organismal dysfunction as well as clinical abnormalities. Lysosomal storage disorders are defined by the type of accumulated substrate, and may be classified as cholesterol storage disorders, sphingolipidoses, oligosaccharidoses, mucolipidoses, mucopolysaccharidoses, lipoprotein storage disorders, neuronal ceroid lipofuscinoses, and others. In some cases, lysosomal storage disorders also include deficiencies or defects in proteins that result in accumulation of macromolecules, such as proteins necessary for normal post-translational modification of lysosomal enzymes, or proteins important for proper lysosomal trafficking. Non-limiting examples of diseases that may be caused by or linked to PGRN deficiency and resulting lysosomal storage disorders include frontotemporal dementia (FTD) (e.g. GRN-FTD, MAPT-FTD, C9ORF72-ALS/FTD), atherosclerosis, Gaucher's disease, Alzheimer's disease (AD), Late onset AD, neuronal ceroid lipofuscinosis (NCL), Niemann-Pick disease type A (NPA), Niemann-Pick disease type B (NPB), Niemann-Pick disease type C (NPC), C9ORF72-associated amyotrophic lateral sclerosis (ALS)/FTD, Parkinson's disease, and age-related macular degeneration (AMD).

Frontotemporal dementia (FTD) is a progressive neurodegenerative disorder. FTD includes a spectrum of clinically, pathologically, and genetically heterogeneous diseases presenting selective involvement of the frontal and temporal lobes (Gazzina et al., Eur J Pharmacol. 817:76-85, 2017). Clinical manifestations of FTD include alterations in behavior and personality, frontal executive deficits, and language dysfunction. Based on the diversity of clinical phenotypes, different presentations have been identified, such as behavioral variants of FTD (bvFTD) and primary progressive aphasia (PPA), which can either be the nonfluent/agrammatic variant PPA (avPPA) or the semantic variant PPA (svPPA). These clinical presentations can also overlap with atypical parkinsonism, such as corticobasal syndrome (CBS), progressive supranuclear palsy (PSP), and amyotrophic lateral sclerosis (ALS) (Gazzina et al., Eur J Pharmacol. 817:76-85, 2017). FTD is associated with various neuropathological hallmarks, including tau pathology in neurons and astrocytes or cytoplasmic ubiquitin inclusions in neurons. The Trans-activating DNA-binding Protein with a molecular weight of 43 kDa (TDP-43) is the most prominent, ubiquitinated protein pathology accumulating in the majority of cases of FTD as well as in ALS (Petkau and Leavitt, Trends Neurosci. 37(7):388-98, 2014). FTD is a significant cause of early-onset dementia with up to 80% of cases presenting between ages 45 and 64. The disease also presents a significant familial component, with about 30-50% of cases reporting family history of the disease (Petkau and Leavitt, supra).

While several genes have been linked to FTD, one of the most frequently mutated genes in FTD is GRN, which maps to human chromosome 17q21 and encodes the cysteine-rich protein PGRN (also known as proepithelin and acrogranin). Recent estimates suggest that GRN mutations account for 5-20% of FTD patients with positive family history and 1-5% of sporadic cases (Rademakers et al., supra). The precise molecular and cellular mechanisms underlying neurodegeneration and disease processes in GRN-associated FTD are unknown, although phenotypic characterization of Grn knockout mice combined with histological analyses of patients' brain suggests that both inflammation and lysosomal defects are central to the disease (Kao et al., Nat Rev Neurosci. 18(6):325-333, 2017). Indeed, massive gliosis is present in cortical regions of patients (Lui et al., Cell. 165(4):921-35, 2016) and lipofuscin, a lysosomal pigment denoting lysosomal disorder, has been reported in the eye and cortex of mutated GRN carriers including both presymptomatic individuals and patients (Ward et al., Sci. Transl. Med. 9(385), 2017).

More than seventy GRN disease mutations have been reported and mapped throughout the gene, where they result in confirmed or predicted loss of function (LOF) alleles (Ji et al. J Med Genet. 54:145-154, 2017). Most heterozygous mutations linked to FTD cause about 50% reduction in mRNA levels primarily as a result of non-sense mRNA decay and a comparable reduction in PGRN protein levels (Petkau and Leavitt, supra; Kao et al., supra). Lower levels of PGRN are also found in the blood (serum) and cerebrospinal fluid (CSF) of carriers, including presymptomatic individuals (Finch et al., Nat Rev Neurosci. 18(6):325-333, 2017; Goossens et al., Alzheimers Res. Ther. 10(1):31, 2018; Meeter et al., Dement. Geriatr. Cogn. Dis. Extra. 6(2):330-340, 2016). Therefore, haploinsufficiency is believed to be the main disease mechanism in GRN-associated FTD, suggesting that therapeutic approaches that elevate PGRN levels in carriers may delay the age of onset as well as the progression of FTD (Petkau and Leavitt, supra; Kao et al., supra). This notion is supported by human genetic studies indicating that a variant of the gene TMEM106B both enhances the levels of PGRN by 25% and delay the age of onset of GRN-associated FTD by 13 years (Nicholson and Rademakers, Acta Neuropathol. 132(5):639-651, 2016).

Homozygous GRN mutations have also been reported, although carriers present a vastly different clinical phenotype known as neuronal ceroid lipofuscinosis (NCL) (Batten disease; incidence 1-2.5 in 100,000 live births; Cotman et al., Curr. Neurol. Neurosci. Rep. 13(8):366, 2013), which is a lysosomal storage disorder (Smith et al., Am. J. Hum. Genet. 90(6):1102-7, 2012; Almeida et al., Neurobiol. Aging. 41:200.e1-200.e5, 2016). GRN is in fact one of the 14 ceroid-lipofuscinosis neuronal (CLN) genes reported to be linked to NCL and GRN is also known as CLN11 (Kollmann et al., Biochim. Biophys. Acta. 1832(11):1866-81, 2013).

Patients with Gaucher's disease who carry homozygous mutations in the GBA gene have lower levels of PGRN in their serum (Jian et al., EBioMedicine 11:127-137, 2016). Parkinson's disease patients with heterozygous mutations in GBA may also have lower levels of PGRN.

Variants in GRN have been linked to AD (Rademakers et al., supra; Brouwers et al., Neurology. 71(9):656-64, 2008; Lee et al., Neurodegener. Dis. 8(4):216-20, 2011; Viswanathan et al., Am. J. Med. Genet. B. Neuropsychiatr. Genet. 150B (5):747-50, 2009) and the TDP-43 pathology is common in the brain of AD patients (Youmans and Wolozin, Exp. Neurol. 237(1):90-5, 2012). PGRN gene delivery has also been shown to decrease amyloid burden in mouse models of AD (van Kampen and Kay, PLOS One. 12(8):e0182896, 2017).

Niemann-Pick disease types A and B (NPA and NPB) result from mutations in the gene encoding acid sphingomyelinase (SMPD1). Niemann-Pick disease type C (NPC) results from mutations in the genes involved in cholesterol transport, i.e., NPC1 and NPC2 (Kolter and Sandhoff, Annu. Rev. Cell Dev. Biol. 21:81-103, 2005; Kobayashi et al., Nat. Cell Biol. 1(2):113-8, 1999).

The vast majority of ALS cases present the TDP-43 pathology, which is also shared with patients harboring GRN mutations (Petkau and Leavitt, Trends Neurosci. 37(7):388-98, 2014; Rademakers et al., Nat. Rev. Neurol. 8(8):423-34, 2012). Among all ALS cases, GGGGCC repeat expansions within the C9ORF72 gene are the most common cause of ALS and a significant cause of FTD. The average mutation frequencies reported in North American and European populations are 37% for familial ALS, 6% for sporadic ALS, 21% for familial FTD, and 6% for sporadic FTD patients (Rademakers et al., supra). Additionally, the TMEM106B variant that is protective in GRN-associated FTD is also protective in FTD patients harboring repeat expansions in the C9ORF72 gene (van Blitterswijk et al., Acta Neuropathol. 127(3):397-406, 2014).

AMD is a degenerative disease and a major cause of blindness in the developed world. It causes damage to the macula, a small spot near the center of the retina and the part of the eye needed for sharp, central vision. The degenerative changes in the eye and loss of vision may be caused by impaired function of lysosomes and harmful protein accumulations behind the retina (Viiri et al., PLOS One. 8(7):e69563, 2013). As the disease progresses, retinal sensory cells in the central vision area are damaged, leading to loss of central vision.

### Kits

In another aspect, the present disclosure provides kits for use in monitoring PGRN levels in a subject (e.g., a test subject and/or a reference subject or population of reference subjects). In some embodiments, the kits are for use in measuring or calibrating the abundance of one or more bis(monoacylglycero)phosphate (BMP) species (e.g., in a test sample obtained from a subject and/or a reference sample obtained from a reference subject or a population of reference subjects). In some embodiments, the kits comprise a BMP standard (e.g., an internal BMP standard) that can be used for measuring or calibrating the abundance of the one or more BMP species (e.g., in a sample such as a test and/or reference sample). In some embodiments, the BMP standard comprises a BMP species that is not naturally present in the subject. In some embodiments, the BMP standard comprises a BMP species that is not naturally present in humans, non-human primates, rodents, dogs, and/or pigs. In some embodiments, the BMP standard comprises a BMP species that is not naturally present in humans. In some embodiments, the BMP standard comprises BMP(14:0_14:0).

In some embodiments, the kit further comprises one or more reagents. For example, in some embodiments, the kit comprises reagents for obtaining a test sample (e.g., from the subject) and/or a reference sample (e.g., from a reference subject or population of reference subjects), processing a sample (e.g., isolating or purifying one or more BMP species from a test sample and/or a reference sample), measuring the abundance of one or more BMP species in a sample (e.g., a test sample and/or a reference sample), and/or calibrating the abundance of one or more BMP species in a sample (e.g., a test sample and/or a reference sample).

In some embodiments, the kit further comprises instructional materials containing directions (e.g., protocols) for the practice of the methods described herein (e.g., instructions for using the kit of monitoring PGRN levels in a subject (e.g., a test subject and/or a reference subject or population of reference subjects)). While the instructional materials typically comprise written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD-ROM), and the like. Such media may include addresses to internet sites that provide such instructional materials.

### EXAMPLES

The following examples are offered for illustrative purposes only, and are not intended to limit the invention in any manner.

### Sample Preparation

Cells (e.g., bone marrow-derived macrophages (BMDMs)) were washed thoroughly with PBS, and BMP species were extracted with methanol spiked with BMP(14:0_14:0) as an internal standard. Following extraction of BMP species with methanol, samples were vortex mixed and centrifuged at 14,000 rpm and 4° C. for 20 minutes. Supernatants were then transferred to liquid chromatography-mass spectrometry vials for further analysis.

Tissue samples were weighed (e.g., 20 mg) and then homogenized in methanol (200 μL) spiked with BMP(14:0_14:0) using a TissueLyser homogenizer (Qiagen, Valencia, CA, USA). Homogenates were spun at 14,000 rpm for 20 minutes at 4° C. Supernatants were then transferred to liquid chromatography-mass spectrometry vials for further analysis.

Biofluids (10 μL) were protein-precipitated with methanol (100 μL) containing BMP(14:0_14:0) and spun at 14,000 rpm for 20 min, 4° C. Supernatants were then transferred to liquid chromatography-mass spectrometry vials for further analysis.

### Lipidomics Procedures

Overall procedures: Animal groups and sample collection were randomized during lipid extraction.

Extraction of lipids and metabolites from urine: Urine was collected into Thermo Matrix Tubes and stored at −80° C. Urine was thawed on ice and centrifuged at 1,000× g for 10 minutes at 4° C. to remove particulates. 10 μL of urine was transferred into 2.0 mL Safe-Lock Eppendorf tube (Eppendorf Cat #022600044) and 200 μL of ice-cold MS-grade methanol containing internal standard mix (2 μL per sample). Samples were vortexed for 5 minutes at 2,500 rpm. Samples were centrifuged for 20 minutes at 21,000× g at 4° C. Methanol supernatant was transferred to LCMS glass vials in 96 well plate. Samples were stored at −80° C. until run on LCMS.

Extraction of lipids and metabolites from plasma: Plasma samples were thawed on ice. Plasma/serum (10 μL) or urine (20 μL) were transferred into a 2 mL Safe-Lock Eppendorf tube (Eppendorf Cat #022600044). Ice cold MS-grade methanol (200 μL) containing internal standard mix (2 μL per sample) was vortexed for 5 min and then centrifuged for 20 min at 21,000× g at 4° C. Methanol supernatant was transferred into LCMS glass vials in 96 well-plates for lipidomics and metabolomics analysis. Samples were stored at −80° C. until run on LCMS.

Extraction of lipids and metabolites from brain: Frontal cortex of mouse brain (18-20 mg) was transferred into 2 mL Safe-Lock Eppendorf tube (Eppendorf Cat #022600044) that was kept in dry ice containing a 5 mm stainless steel bead (QIAGEN Cat #69989). MS-grade methanol (400 μL) containing internal standard mix (2 μL per sample) was added. Tissues were homogenized with Tissuelyser for 30 sec at 25 Hz in the cold room and then centrifuged for 20 min at 21,000× g at 4° C. (bead left in the tube). Methanol supernatant was transferred into new 1.5 mL Eppendorf vials and left at −20° C. for 1 hour to allow further precipitation of proteins. Vials were centrifuged for 20 min at 21,000× g at 4° C. and the methanol supernatant was transferred in LCMS glass vials for lipidomics and metabolomics analysis. Samples stored at −80° C. until run on LCMS.

Extraction of lipids and metabolites from CSF: CSF was collected from mouse with the pipet method. CSF (5 μL) was added to ice-cold MS-grad MeOH (100 μL) containing internal standard mix (0.2 μL per sample) directly in glass vials. Glass vials with CSF and MeOH were vortexed for 5 minutes and directly run on LCMS.

Extraction of lipids and metabolites from liver: liver (20 mg) was transferred into a 2 mL Safe-Lock Eppendorf tube (Eppendorf Cat #022600044) kept in dry ice containing a 5 mm stainless steel bead (QIAGEN Cat #69989). MS-grade methanol (400 μL) containing internal standard mix (2 μL per sample) was then added. Tissues were homogenized with Tissuelyser for 30 sec at 25 Hz (in the cold room) and centrifuged for 20 min at 21,000× g at 4° C. (bead left in the tube). The methanol supernatant was transferred to new 1.5 mL Eppendorf vials and left at −20° C. for three hours to allow further precipitation of proteins. Vials were centrifuged for 20 min at 21,000× g at 4° C., and the methanol supernatant was transferred to LCMS glass vials for lipidomics and metabolomics analysis. Samples stored −80° C. until run on LCMS.

### Liquid Chromatography-Mass Spectrometry

BMP analyses were performed by liquid chromatography (Shimadzu Nexera X2 system, Shimadzu Scientific Instrument, Columbia, MD, USA) coupled to electrospray mass spectrometry (Sciex 6500+QTRAP, Sciex, Framingham, MA, USA). For each analysis, 5 μL of sample was injected onto a BEH amide 1.7 μm, 2.1×150 mm column (Waters Corporation, Milford, Massachusetts, USA) using a flow rate of 0.40 mL/min. at 55° C. Mobile phase A consisted of water with 10 mM ammonium formate+0.1% formic acid. Mobile phase B consisted of acetonitrile with 0.1% formic acid. The gradient was programmed as follows: 0.0-1.0 min. at 95% B; 1.0-7.0 min. to 50% B; 7.0-7.1 min. to 95% B; and 7.1-12.0 min. at 95% B. Electrospray ionization was performed in the negative-ion mode using the following settings: curtain gas at 25; collision gas was set at medium; ion spray voltage at −4500; temperature at 600; ion source gas 1 at 50; ion source gas 2 at 60; collision energy at −50, CXP at −15; DP at −60; EP at −10; dwell time at 20 ms. Data acquisition was performed using Analyst 1.6.3 (Sciex) in multiple reaction monitoring mode (MRM). BMP species were detected using the MRM transition parameters reported in Table 1. BMP species were quantified using BMP(14:0_14:0) as the internal standard. BMP species were identified based on their retention times and MRM properties. Quantification was performed using MultiQuant 3.02 (Sciex) after correction for isotopic overlap. BMP species were normalized to either total protein amount, tissue weight or biofluid volume. Protein concentration was measured using the bicinchoninic acid (BCA) assay (Pierce, Rockford, IL, USA).

Precursor (Q1) [M−H]⁻ and product ion (Q3) m/z transitions were used to measure BMP species. The BMP species were identified from the Q1 and Q3 values according to Table 1. Abbreviations are used herein to refer to species with two side-chains, where the structures of the fatty acid side chains are indicated within parentheses in the BMP format (e.g., BMP(18:1_18:1)). The numerals follow the standard fatty acid notation format of number of fatty acid carbon atoms: number of double bonds. The “e-” prefix is used to indicate the presence of an alkyl ether substituent (e.g., BMP(16:0e_18:0)) where the carbonyl oxygen of the fatty acid side chain is replaced with two hydrogen atoms. Alternatively the BMP species can be referred to generically according to the total number of carbon atoms: total number of double bonds; species having similar values can be distinguished by their Q1 and Q3 values.

TABLE 1
BMP species and MRM transition parameters

| | Total carbon | | |
|---|---|---|---|
| | atoms:total | | |
| Name | unsaturations | Q1 | Q3 |
| BMP(14:0_14:0) | BMP(28:0) | 665.5 | 227.2 |
| BMP(14:0_16:0) | BMP(30:0) | 693.6 | 255.2 |
| BMP(14:0_16:1) | BMP(30:1) | 691.6 | 253.2 |
| BMP(14:0_18:0) | BMP(32:0) | 721.6 | 283.2 |
| BMP(14:0_18:1) | BMP(32:1) | 719.6 | 281.2 |
| BMP(14:0_18:2) | BMP(32:2) | 717.6 | 279.2 |
| BMP(14:0_18:3) | BMP(32:3) | 715.6 | 277.2 |
| BMP(14:0_20:1) | BMP(34:1) | 747.6 | 309.2 |
| BMP(14:0_20:2) | BMP(34:2) | 745.6 | 307.2 |
| BMP(14:0_20:3) | BMP(34:3) | 743.6 | 305.2 |
| BMP(14:0_20:4) | BMP(34:4) | 741.6 | 303.2 |
| BMP(14:0_20:5) | BMP(34:5) | 739.6 | 301.2 |
| BMP(14:0_22:4) | BMP(36:4) | 769.6 | 331.2 |
| BMP(14:0_22:5) | BMP(36:5) | 767.6 | 329.2 |
| BMP(14:0_22:6) | BMP(36:6) | 765.6 | 327.2 |
| BMP(16:0_16:0) | BMP(32:0) | 721.6 | 255.2 |
| BMP(16:0_16:1) | BMP(32:1) | 719.6 | 253.2 |
| BMP(16:0_18:0) | BMP(34:0) | 749.6 | 283.2 |
| BMP(16:0_18:1) | BMP(34:1) | 747.6 | 281.2 |
| BMP(16:0_18:2) | BMP(34:2) | 745.6 | 279.2 |
| BMP(16:0_18:3) | BMP(34:3) | 743.6 | 277.2 |
| BMP(16:0_20:1) | BMP(36:1) | 775.6 | 309.2 |
| BMP(16:0_20:2) | BMP(36:2) | 773.6 | 307.2 |
| BMP(16:0_20:3) | BMP(36:3) | 771.6 | 305.2 |
| BMP(16:0_20:4) | BMP(36:4) | 769.6 | 303.2 |
| BMP(16:0_20:5) | BMP(36:5) | 767.6 | 301.2 |
| BMP(16:0_22:4) | BMP(38:4) | 797.6 | 331.2 |
| BMP(16:0_22:5) | BMP(38:5) | 795.6 | 329.2 |
| BMP(16:0_22:6) | BMP(38:6) | 793.6 | 327.2 |
| BMP(16:1_16:1) | BMP(32:2) | 717.6 | 253.2 |
| BMP(16:1_18:0) | BMP(34:1) | 747.6 | 283.2 |
| BMP(16:1_18:1) | BMP(34:2) | 745.6 | 281.2 |
| BMP(16:1_18:2) | BMP(34:3) | 743.6 | 279.2 |
| BMP(16:1_18:3) | BMP(34:4) | 741.6 | 277.2 |
| BMP(16:1_20:1) | BMP(36:2) | 773.6 | 309.2 |
| BMP(16:1_20:2) | BMP(36:3) | 771.6 | 307.2 |
| BMP(16:1_20:3) | BMP(36:4) | 769.6 | 305.2 |
| BMP(16:1_20:4) | BMP(36:5) | 767.6 | 303.2 |
| BMP(16:1_20:5) | BMP(36:6) | 765.6 | 301.2 |
| BMP(16:1_22:4) | BMP(38:5) | 795.6 | 331.2 |
| BMP(16:1_22:5) | BMP(38:6) | 793.6 | 329.2 |
| BMP(16:1_22:6) | BMP(38:7) | 791.6 | 327.2 |
| BMP(16:0e_14:0) | BMP(40:0) | 679.5 | 227.2 |
| BMP(16:0e_16:0) | BMP(32:0) | 707.6 | 255.2 |
| BMP(16:0e_18:0) | BMP(34:0) | 735.6 | 283.2 |
| BMP(16:0e_18:1) | BMP(34:1) | 733.6 | 281.2 |
| BMP(16:0e_18:2) | BMP(34:2) | 731.6 | 279.2 |
| BMP(16:0e_18:3) | BMP(34:3) | 729.6 | 277.2 |
| BMP(16:0e_20:3) | BMP(36:3) | 757.6 | 305.2 |
| BMP(16:0e_20:4) | BMP(36:4) | 755.6 | 303.2 |
| BMP(16:0e_20:5) | BMP(36:5) | 753.6 | 301.2 |
| BMP(16:0e_22:4) | BMP(38:4) | 783.6 | 331.2 |
| BMP(16:0e_22:6) | BMP(38:6) | 779.6 | 327.2 |
| BMP(16:1e_14:0) | BMP(30:1) | 677.5 | 227.2 |
| BMP(16:1e_16:0) | BMP(32:1) | 705.6 | 255.2 |
| BMP(16:1e_18:0) | BMP(34:1) | 733.6 | 283.2 |
| BMP(16:1e_18:1) | BMP(34:2) | 731.6 | 281.2 |
| BMP(16:1e_18:2) | BMP(34:3) | 729.6 | 279.2 |
| BMP(16:1e_18:3) | BMP(34:4) | 727.6 | 277.2 |
| BMP(16:1e_20:3) | BMP(36:4) | 755.6 | 305.2 |
| BMP(16:1e_20:4) | BMP(36:5) | 753.6 | 303.2 |
| BMP(16:1e_20:5) | BMP(36:6) | 751.6 | 301.2 |
| BMP(16:1e_22:4) | BMP(38:5) | 781.6 | 331.2 |
| BMP(16:1e_22:6) | BMP(38:7) | 777.6 | 327.2 |
| BMP(18:0_18:0) | BMP(36:0) | 777.6 | 283.2 |
| BMP(18:0_18:1) | BMP(36:1) | 775.6 | 281.2 |
| BMP(18:0_18:2) | BMP(36:2) | 773.6 | 279.2 |
| BMP(18:0_18:3) | BMP(36:3) | 771.6 | 277.2 |
| BMP(18:0_20:1) | BMP(38:1) | 803.6 | 309.2 |
| BMP(18:0_20:2) | BMP(38:2) | 801.6 | 307.2 |
| BMP(18:0_20:3) | BMP(38:3) | 799.6 | 305.2 |
| BMP(18:0_20:4) | BMP(38:4) | 797.6 | 303.2 |
| BMP(18:0_20:5) | BMP(38:5) | 795.6 | 301.2 |
| BMP(18:0_22:4) | BMP(40:4) | 825.6 | 331.2 |
| BMP(18:0_22:5) | BMP(40:5) | 823.6 | 329.2 |
| BMP(18:0_22:6) | BMP(40:6) | 821.6 | 327.2 |
| BMP(18:1_18:1) | BMP(36:2) | 773.6 | 281.2 |
| BMP(18:1_18:2) | BMP(36:3) | 771.6 | 279.2 |
| BMP(18:1_18:3) | BMP(36:4) | 769.6 | 277.2 |
| BMP(18:1_20:1) | BMP(38:2) | 801.6 | 309.2 |
| BMP(18:1_20:2) | BMP(38:3) | 799.6 | 307.2 |
| BMP(18:1_20:3) | BMP(38:4) | 797.6 | 305.2 |
| BMP(18:1_20:4) | BMP(38:5) | 795.6 | 303.2 |
| BMP(18:1_20:5) | BMP(38:6) | 793.6 | 301.2 |
| BMP(18:1_22:4) | BMP(40:5) | 823.6 | 331.2 |
| BMP(18:1_22:5) | BMP(40:6) | 821.6 | 329.2 |
| BMP(18:1_22:6) | BMP(40:7) | 819.6 | 327.2 |
| BMP(18:2_18:2) | BMP(36:4) | 769.6 | 279.2 |
| BMP(18:2_18:3) | BMP(36:5) | 767.6 | 277.2 |
| BMP(18:2_20:1) | BMP(38:3) | 799.6 | 309.2 |
| BMP(18:2_20:2) | BMP(38:4) | 797.6 | 307.2 |
| BMP(18:2_20:3) | BMP(38:5) | 795.6 | 305.2 |
| BMP(18:2_20:4) | BMP(38:6) | 793.6 | 303.2 |
| BMP(18:2_20:5) | BMP(38:7) | 791.6 | 301.2 |
| BMP(18:2_22:4) | BMP(40:6) | 821.6 | 331.2 |
| BMP(18:2_22:5) | BMP(40:7) | 819.6 | 329.2 |
| BMP(18:2_22:6) | BMP(40:8) | 817.6 | 327.2 |
| BMP(18:3_18:3) | BMP(36:6) | 765.6 | 277.2 |
| BMP(18:3_20:1) | BMP(38:4) | 797.6 | 309.2 |
| BMP(18:3_20:2) | BMP(38:5) | 795.6 | 307.2 |
| BMP(18:3_20:3) | BMP(38:6) | 793.6 | 305.2 |
| BMP(18:3_20:4) | BMP(38:7) | 791.6 | 303.2 |
| BMP(18:3_20:5) | BMP(38:8) | 789.6 | 301.2 |
| BMP(18:3_22:4) | BMP(40:7) | 819.6 | 331.2 |
| BMP(18:3_22:5) | BMP(40:8) | 817.6 | 329.2 |
| BMP(18:3_22:6) | BMP(40:9) | 815.6 | 327.2 |
| BMP(18:0e_14:0) | BMP(32:0) | 707.5 | 227.2 |
| BMP(18:0e_16:0) | BMP(34:0) | 735.6 | 255.2 |
| BMP(18:0e_18:0) | BMP(36:0) | 763.6 | 283.2 |
| BMP(18:0e_18:1) | BMP(36:1) | 761.6 | 281.2 |
| BMP(18:0e_18:2) | BMP(36:2) | 759.6 | 279.3 |
| BMP(18:0e_18:3) | BMP(36:3) | 757.6 | 277.2 |
| BMP(18:0e_20:3) | BMP(38:3) | 785.6 | 305.2 |
| BMP(18:0e_20:4) | BMP(38:4) | 783.6 | 303.2 |
| BMP(18:0e_20:5) | BMP(38:5) | 781.6 | 301.2 |
| BMP(18:0e_22:4) | BMP(40:4) | 811.6 | 331.3 |
| BMP(18:0e_22:6) | BMP(40:6) | 807.6 | 327.3 |
| BMP(18:1e_14:0) | BMP(32:1) | 705.5 | 227.2 |
| BMP(18:1e_16:0) | BMP(34:1) | 733.6 | 255.2 |
| BMP(18:1e_18:0) | BMP(36:1) | 761.6 | 283.2 |
| BMP(18:1e_18:1) | BMP(36:2) | 759.6 | 281.2 |
| BMP(18:1e_18:2) | BMP(36:3) | 757.6 | 279.3 |
| BMP(18:1e_18:3) | BMP(36:4) | 755.6 | 277.2 |
| BMP(18:1e_20:3) | BMP(38:4) | 783.6 | 305.2 |
| BMP(18:1e_20:4) | BMP(38:5) | 781.6 | 303.2 |
| BMP(18:1e_20:5) | BMP(38:6) | 779.6 | 301.2 |
| BMP(18:1e_22:4) | BMP(40:5) | 809.6 | 331.3 |
| BMP(18:1e_22:6) | BMP(40:7) | 805.6 | 327.3 |
| BMP(20:3_20:3) | BMP(40:6) | 821.6 | 305.2 |
| BMP(20:3_20:4) | BMP(40:7) | 819.6 | 303.2 |
| BMP(20:3_20:5) | BMP(40:8) | 817.6 | 301.2 |
| BMP(20:3_22:4) | BMP(42:7) | 847.6 | 331.3 |
| BMP(20:3_22:5) | BMP(42:8) | 845.6 | 329.3 |
| BMP(20:3_22:6) | BMP(42:9) | 843.6 | 327.3 |
| BMP(20:4_20:4) | BMP(40:8) | 817.6 | 303.2 |
| BMP(20:4_20:5) | BMP(40:9) | 815.6 | 301.2 |
| BMP(20:4_22:4) | BMP(42:8) | 845.6 | 331.3 |
| BMP(20:4_22:5) | BMP(42:9) | 843.6 | 329.3 |
| BMP(20:4_22:6) | BMP(42:10) | 841.6 | 327.3 |
| BMP(20:5_20:5) | BMP(40:10) | 813.6 | 301.2 |
| BMP(20:5_22:4) | BMP(42:9) | 843.6 | 331.3 |
| BMP(20:5_22:5) | BMP(42:10) | 841.6 | 329.3 |
| BMP(20:5_22:6) | BMP(42:11) | 839.6 | 327.3 |
| BMP(22:4_22:4) | BMP(44:8) | 873.6 | 331.3 |
| BMP(22:4_22:5) | BMP(44:9) | 871.6 | 329.3 |
| BMP(22:4_22:6) | BMP(44:10) | 869.6 | 327.3 |
| BMP(22:6_22:6) | BMP(44:12) | 865.6 | 327.2 |

### Example 1. Expression and Isolation of Recombinant Fusion Proteins

To express the recombinant Fc dimer: PGRN fusion proteins in Expi293 (Thermo-Fisher), cells were transfected at 2×10⁶ cells/mL density with Expifectamine™ 293/plasmid DNA complex according to manufacturer's instructions (Thermo-Fisher). After transfection, cells were incubated at 37° C. with a humidified atmosphere of 6-8% CO₂ in an orbital shaker (Infors HT Multitron). On day one post-transfection, Expifectamine™ transfection enhancer 1 and 2 were added to the culture. Media supernatant was harvested by centrifugation after 96-hour post-transfection. The clarified supernatant was supplemented with EDTA-free protease inhibitor (Roche) and was stored at −80° C.

For recombinant fusion protein isolation, clarified media supernatant was loaded on HiTrap MabSelect SuRe Protein A affinity column (GE Healthcare Life Sciences) and washed with wash buffer I (PBS buffer pH 7.4) and wash buffer II (PBS buffer pH 7.4 and 150 mM NaCl). The fusion protein was eluted in 50 mM QB citrate buffer pH 3.0 with 150 mM NaCl. Immediately after elution, the arginine-succinate buffer (1 M arginine, 685 mM succinic acid pH 5.0) was added to adjust the pH. Protein aggregates were separated from monodispersed fusion proteins by size exclusion chromatography (SEC) on Superdex 200 increase 16/60 GL column (GE Healthcare Life Sciences). The SEC mobile phase was kept in arginine-succinate pH 5.0 buffer. All chromatography steps were performed on AKTA pure or AKTA Avant systems (GE Healthcare Life Sciences).

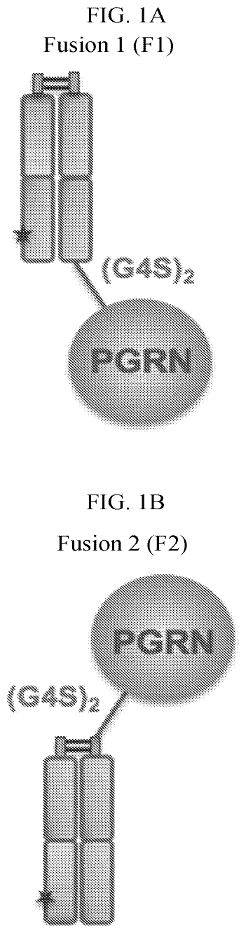

PGRN fusion proteins F1 and F2 are depicted in FIGS. 1A and 1B. In F1, PGRN was fused at its N-terminus via-Gly-Gly-Gly-Gly-Ser-Gly-Gly-Gly-Gly-Ser- ((G4S)₂) (SEQ ID NO:1) to one of the Fc polypeptides. In F2, PGRN was fused at its C-terminus via (G4S)₂ (SEQ ID NO:1) to one of the Fc polypeptides. For both F1 and F2, the Fc polypeptide that was fused to progranulin was covalently linked via disulfide bonds in the hinge region of another Fc polypeptide to form an Fc heterodimer.

### Example 2. Treatment of BMDMs from Grn Knockout Mice

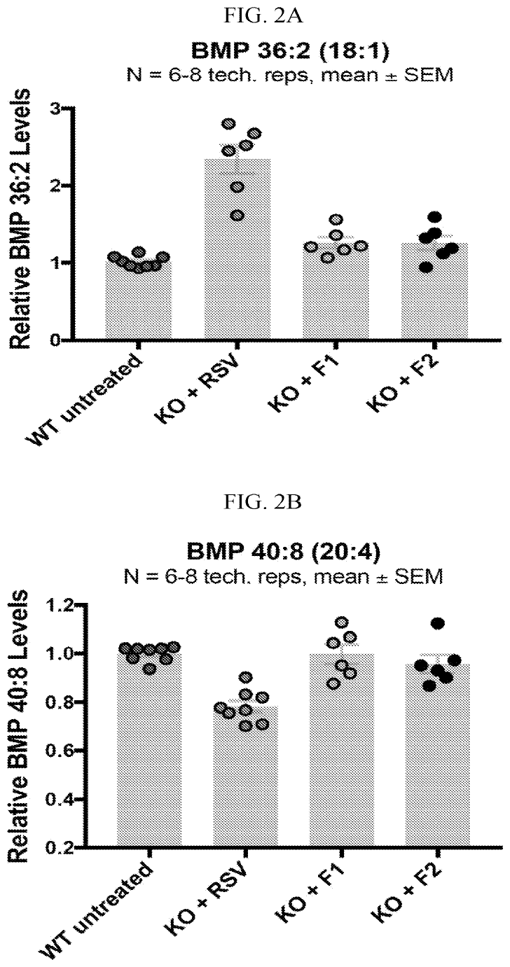

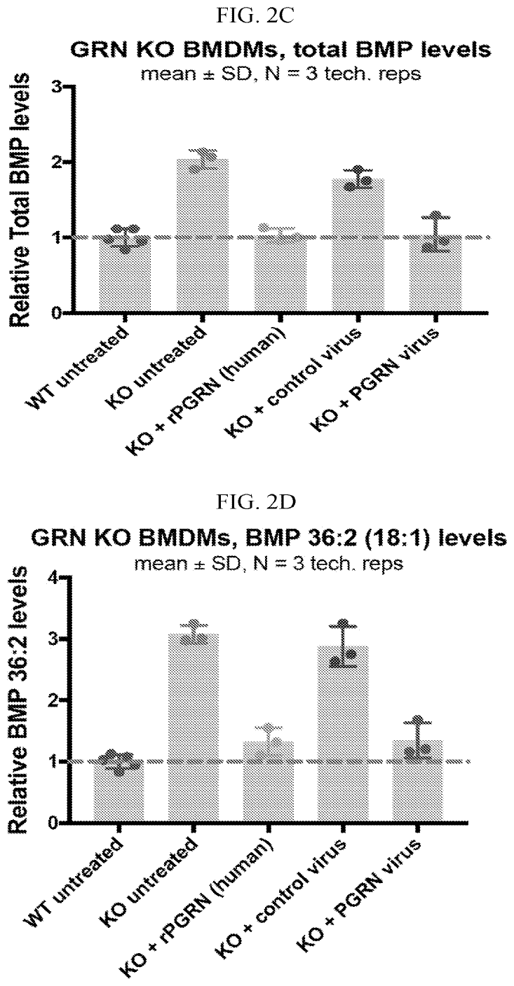

BMDMs were derived in vitro from bone marrow of wild-type and Grn knockout mice using a similar method to that of Trouplin et al. (J. Vis. Exp. 2013(81) 50966), but recombinant M-CSF was added directly to the cell growth media to induce differentiation. The BMDMs were treated for 72 hours with 50 nM PGRN fusion proteins F1 and F2 of Example 1 or RSV (respiratory syncytial virus) control. Cellular lipids were extracted via addition of methanol containing an internal standard mixture and BMP abundance was measured by liquid chromatography-mass spectrometry (LC-MS/MS) on a Q-trap 6500 (SCIEX). As shown in FIGS. 2A and 2B, the abundance of BMP(18:1_18:1) and BMP(20:4_20:4) was increased in BMDMs from untreated Grn knockout mice compared to the wild-type control. Furthermore, in separate experiments (three technical repetitions), as shown in FIGS. 2C and 2D, total BMP and BMP(18:1_18:1) abundance decreased in the Grn knockout BMDMs that were treated with either recombinant PGRN (Adipogen) or PGRN expressed by lentivirus.

BMP species that were found to have increased abundance in BMDMs derived from granulin (GRN) knockout animals are shown in Table 2. Species that exhibited particularly marked increases in abundance are marked with an asterisk.

TABLE 2
Elevated BMP species in BMDM of Grn KO mice
BMP

BMP(16:0_18:1)
BMP(16:0_18:2)
BMP(18:0_18:0)
BMP(18:0_18:1)
BMP(18:1_18:1)*
BMP(16:0_20:3)
BMP(18:1_20:2)
BMP(18:0_20:4)*
BMP(16:0_22:5)
BMP(20:4_20:4)*
BMP(22:6_22:6)*
BMP(20:4_20:5)
BMP(18:2_18:2)
BMP(16:0_20:4)
BMP(18:0_18:2)
BMP(18:0e_22:6)
BMP(18:1e_20:4)
BMP(20:4_22:6)*
BMP(18:0e_20:4)
BMP(18:2_20:4)
BMP(18:1_22:6)*
BMP(18:1_20:4)*
BMP(18:0_22:6)*

### Example 3. Treatment of Grn KO Mice to Rescue Phenotypes

Fusion 1 and Fusion 2 progranulin fusion proteins of Example 1 were injected via the tail vein into Grn WT and Grn KO mice to determine whether peripheral and CNS Grn KO phenotypes could be rescued following a single 50 mg/kg injection.

### Materials and Methods

Animals: The mice used for this study were obtained from JAX Laboratories and consisted of 21 Grn KO mice (n=12 males, n=9 females) and 10 Grn WT mice (n=5 males, n=5 females) age 3-5 months (Table 3). Animals were housed in standard conditions in the vivarium with ad libitum access to food and water at least 7 days prior to the initiation of the study.

TABLE 3
Study Design/Experimental groups

| Genotype | Protein | Time points (h) | n/group |
|---|---|---|---|
| Grn KO | Vehicle | 0.5, 24, 48, 96 | 4 |
| | Vehicle | 0.5, 24 | 4 |
| | Fusion 1 | 0.5, 24, 48, 96 | 5 |
| | Fusion 1 | 0.5, 24 | 4 |
| | Fusion 2 | 0.5, 24, 48, 96 | 4 |
| WT | Vehicle | 0.5, 24, 48, 96 | 5 |
| | Vehicle | 0.5, 24 | 5 |

Animal allocation to experimental groups: Mice were distributed equally in each experimental group to account for differences across litters, gender, and age.

Formulation: Fc dimer:PGRN fusion proteins Fusion 1 and Fusion 2 were used at 5.05 mg/mL and 4.90 mg/mL saline, respectively.

Overall procedures: Experimental conditions were alternated when collecting tissues. Animal groups and sample collections were randomized.

In-life procedures: Submandibular bleeds were performed using 3 mm lancets (GoldenRod animal lancets). Plasma collection: Blood was collected in EDTA tubes (Sarstedt Microvette 500 K3E, Ref #201341102) and slowly inverted 10 times. For small volumes of blood (<100 L), blood was collected in EDTA tubes with capillary tube (Sarstedt Microvette 100 K3E, Ref #201278100). EDTA tubes were immediately stored in the fridge until plasma preparation. The time between storage and preparation did not exceed 1 hour. The time of collection and time of processing were followed consistently. The tubes were centrifuged at 12,700 rpm for 7 minutes at 4° C. Plasma (top layer) was transferred to 0.6 mL Matrix tubes with rubber seal. Matrix tubes were snap frozen on dry ice before transferring to −80° C. Urine collection: Urine was collected by restraining mice in a plastic weigh boat, causing most of them to expel urine into the weigh boat. The urine was collected with a p200 pipet, transferred to 0.6 mL matrix tubes with rubber seal. Matrix tubes were snap frozen on dry ice before transferring to −80° C.

Terminal fluids/tissue collection procedures: Animals were deeply anesthetized via intraperitoneal (i.p.) injection of 2.5% Avertin and then tissues were collected in the order as described below:

Samples collected before intracardiac perfusion: Plasma collection: Blood was collected via cardiac puncture using a 1 mL Terumo tuberculin syringe attached to a 25 gauge needle (Ref #SS-01T2516) (Not pre-conditioned with EDTA). The needle was then detached and the blood was transferred to EDTA tubes (Sarstedt Microvette 500 K3E, Ref #201341102) and slowly inverted 10 times. Following this procedure, post collection methods were continued as described above in In-Life Procedures. Cerebrospinal fluid collection: The three muscle layers on the back of the neck were peeled back with small scissors and forceps and the area surrounding the revealed cisterna magna was cauterized to prevent contamination from blood, and the membrane was cleaned with a Q-tip and PBS. The membrane was dried and the cisterna magna was punctured with the tip of a 28½ gauge insulin syringe needle. A p20 pipet was then quickly placed over the newly punctured hole and CSF drawn up into the pipet tip. CSF was placed in a 0.5 mL Lo-bind Eppendorf tube and spun at 12,700 rpm for 7 minutes at 4° C. The CSF supernatant was transferred to a 0.5 mL Lo-bind Eppendorf tube and snap frozen on dry ice before transferring to −80° C.

For samples collected after intracardiac perfusion, animals were transcardially perfused for 5 minutes with ice cold PBS using a peristaltic pump (Gilson Inc. Minipuls Evolution F110701) at a flow rate of 5 mL/minute. For tissues that could be dissected and pre-weighed during collection, the samples were placed directly into 1.5 mL Eppendorf tubes. Tissues were collected in the following order: Liver (post-perfusion): about 150 mg of liver was collected into 1.5 mL Eppendorf tubes. Eppendorf tubes were snap frozen on dry ice before transferring to −80° C. Eye: both eyes were removed, muscle and optic nerve removed, and eyes placed in a single 1.5 mL Eppendorf tube. Eppendorf tubes were snap frozen on dry ice before transferring to −80° C. Brain: right hemisphere without olfactory bulb and cerebellum was collected and weighed before placing in 1.5 mL Eppendorf tubes. Eppendorf tubes were snap frozen on dry ice before transferring to −80° C.

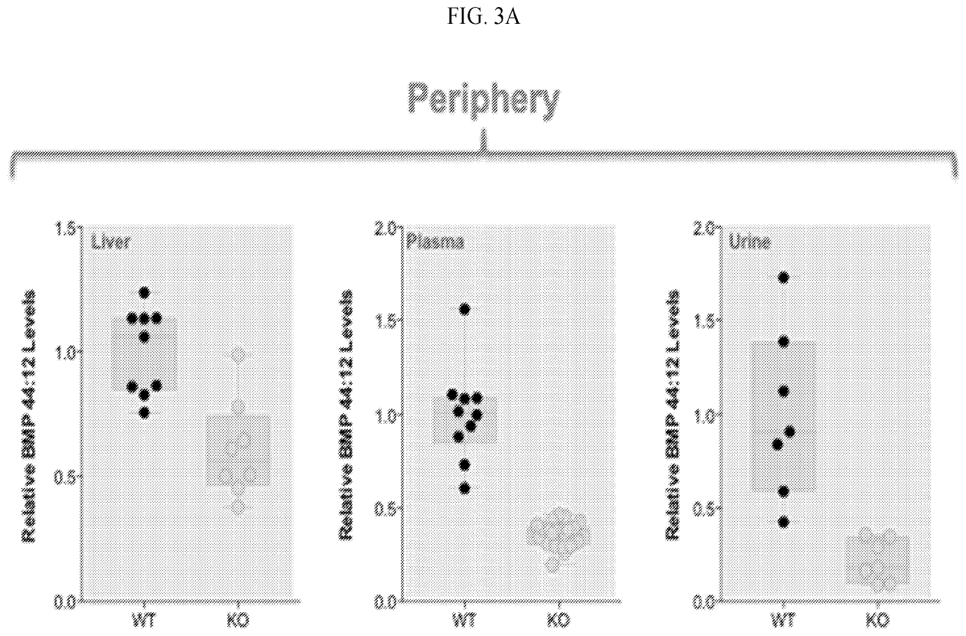

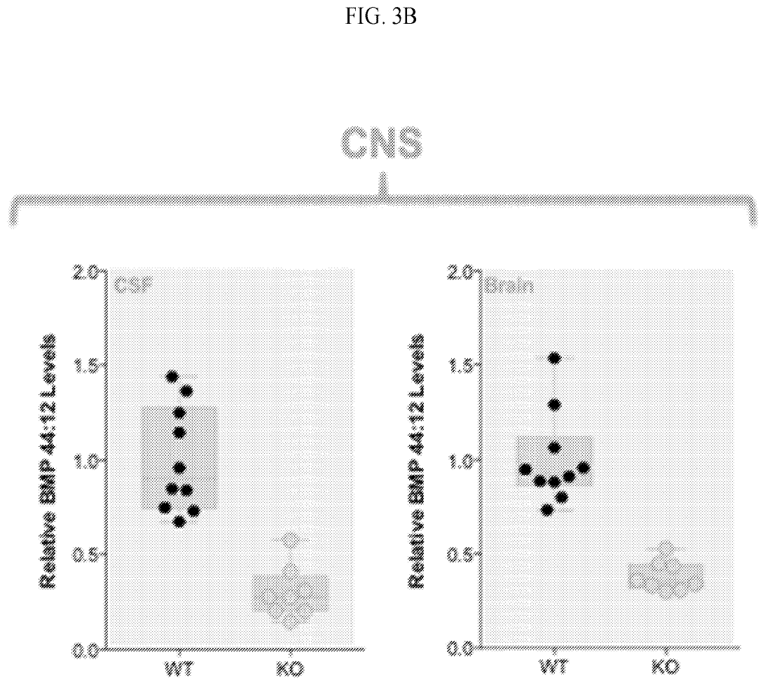

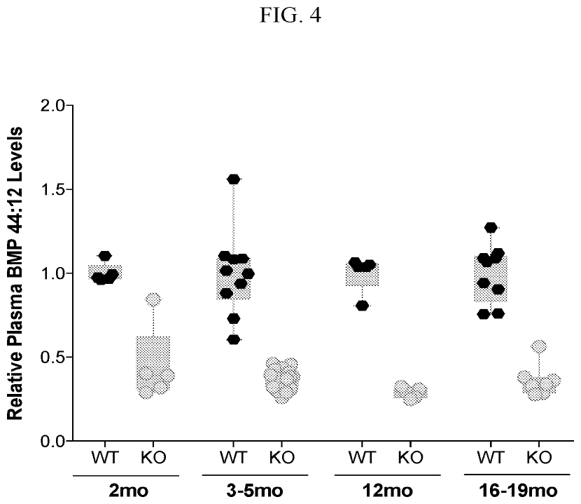

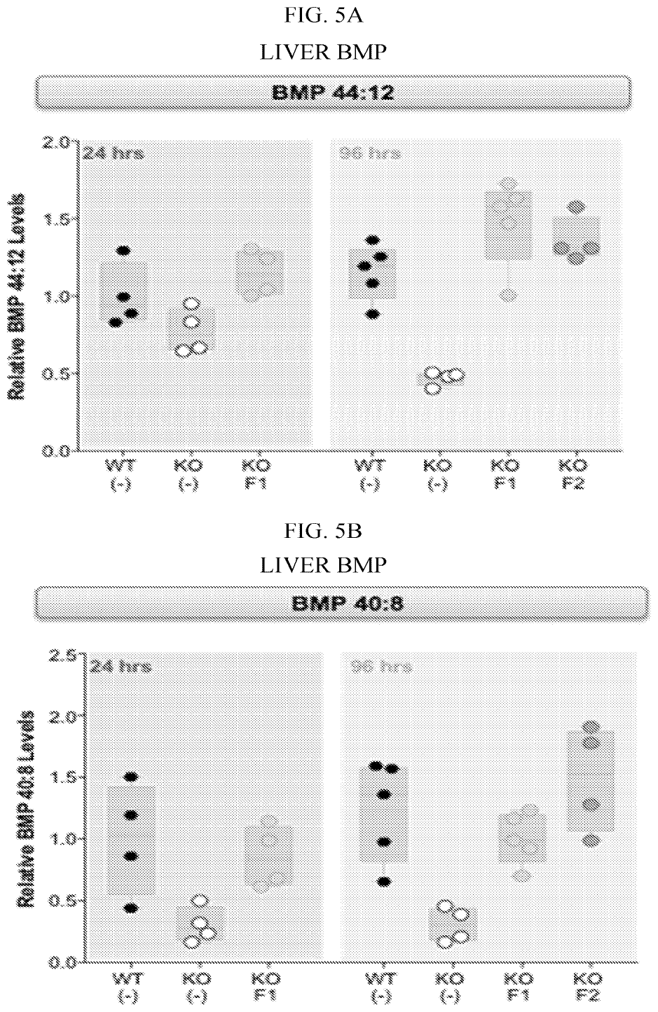

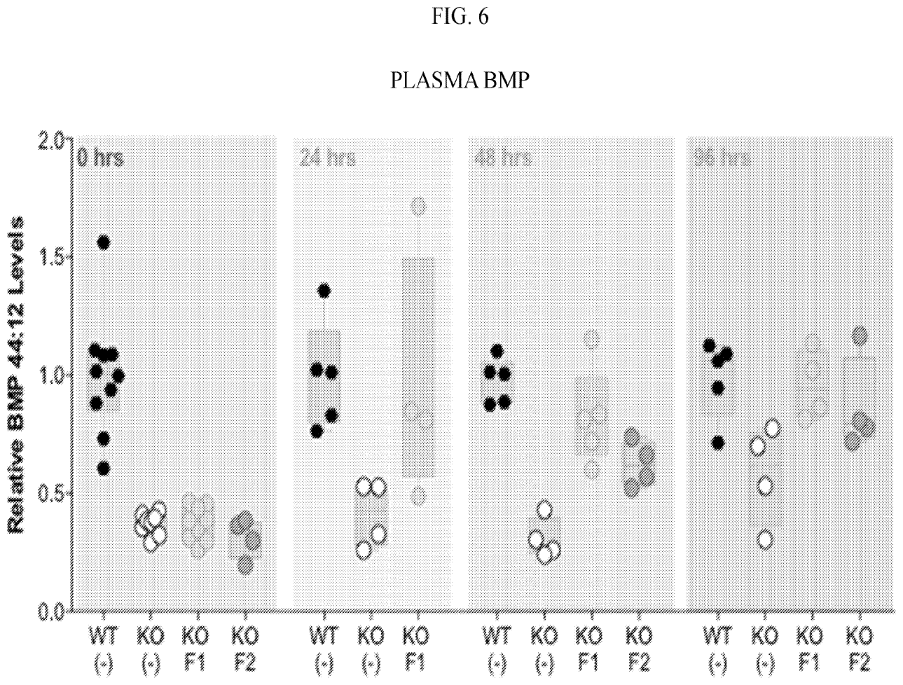

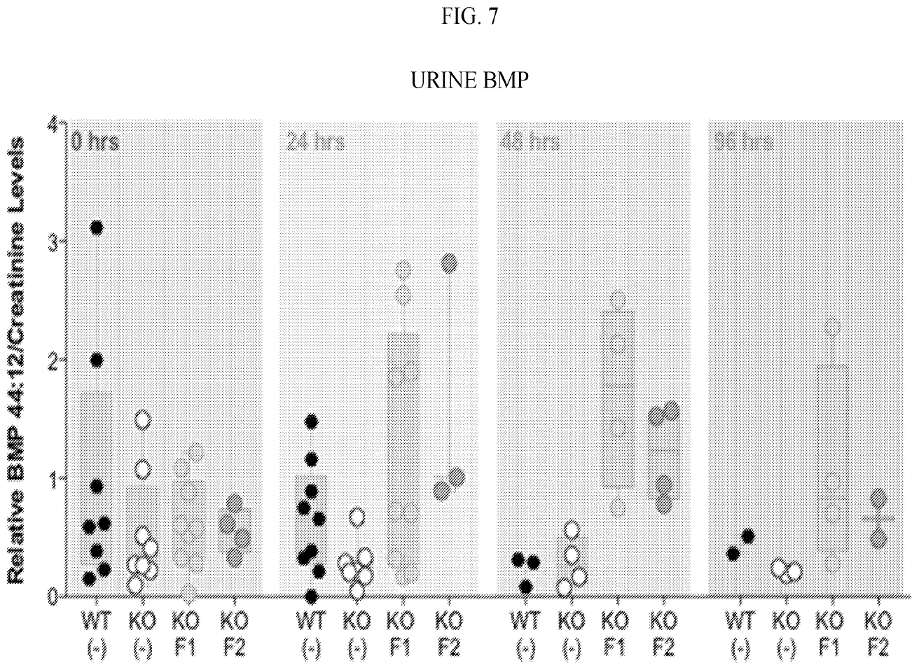

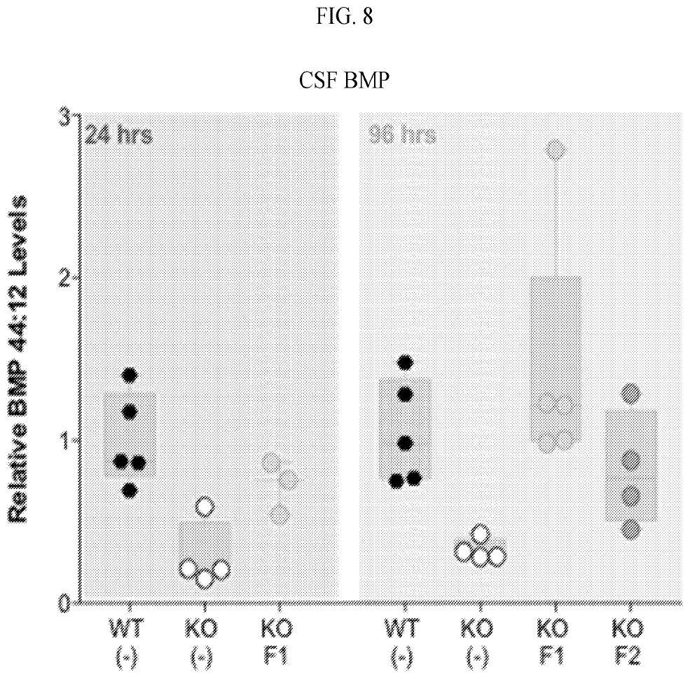

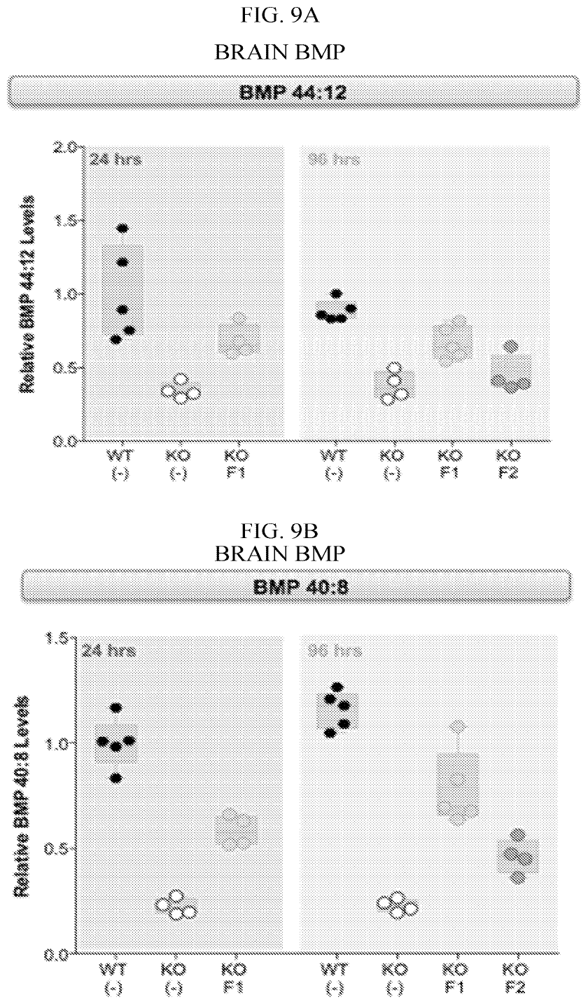

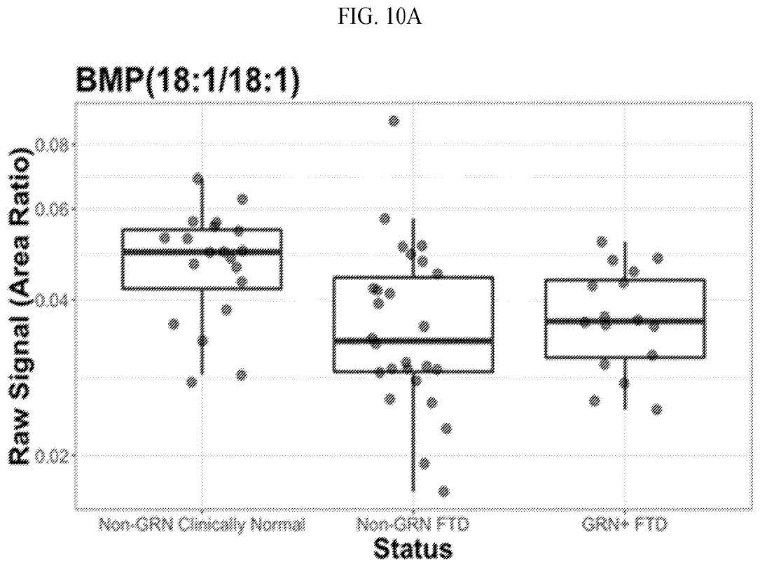

As shown in FIGS. 3-9, administration of PGRN fusion proteins F1 and F2 were found to increase and restore BMP levels in the liver, plasma, urine, CSF, and brain.

### Example 4. BMP Changes in CSF of FTD Patient Samples

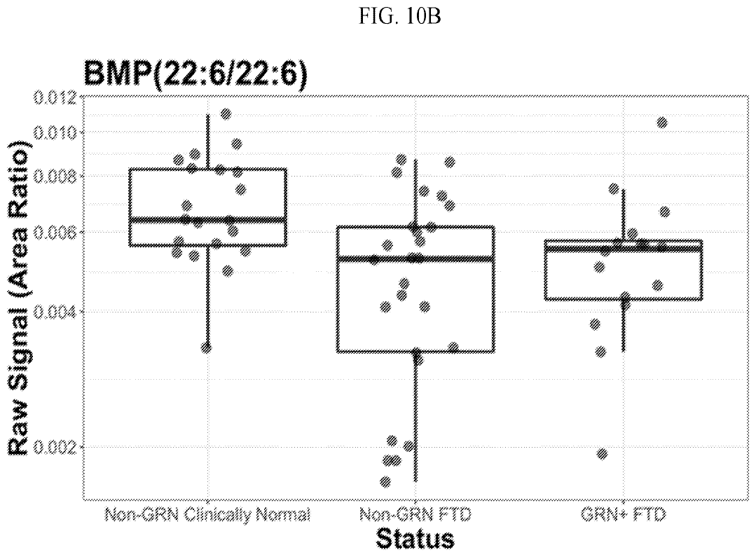

Samples from the National Cell Repository for Alzheimer's Disease (NCRAD), which receives government support under a cooperative agreement grant (U24 AG21886) awarded by the National Institute on Aging (NIA), were used in this study. CSF samples from sporadic FTD patients (n=25), GRN mutant FTD patients (n=16) and clinically normal control subjects (n=20) were blinded, randomized, and subjected to metabolite/lipid extraction by methanol and analyzed on a quantitative LC/MS/MS platform. The analyte identities were confirmed with authentic compounds, and each analyte's signal was normalized to a corresponding internal standard. Decreases in BMP 18:1_18:1 and 22:6_22:6 in CSF of sporadic and GRN FTD patient samples were observed compared to clinically normal controls (FIGS. 10A and 10B).

It is understood that the examples and embodiments described herein are for illustrative purposes only and that various modifications or changes in light thereof will be suggested to persons skilled in the art and are to be included within the spirit and purview of this application and scope of the appended claims. The sequences of the sequence accession numbers cited herein are hereby incorporated by reference.

### INFORMAL SEQUENCE LISTING

| SEQ ID | | |
|---|---|---|
| NO: | Sequence | Description |
| 1 | GGGGSGGGGS | (G4S)₂ linker sequence |
| 2 | GGGGS | G4S |

What is claimed is:

1. A method for evaluating a compound or monitoring a subject's response to a compound, pharmaceutical composition, or dosing regimen thereof for treating a progranulin (PGRN)-associated disorder, the method comprising:
(a) measuring an abundance of one or more bis(monoacylglycero)phosphate (BMP) species in a test sample from a subject having a PGRN-associated disorder, wherein the test sample or subject has been treated with the compound or pharmaceutical composition thereof;
(b) comparing the difference in abundance between the one or more BMP species measured in (a) and one or more reference values; and
(c) determining from the comparison whether the compound, pharmaceutical composition, or dosing regimen thereof improves one or more BMP species levels for treating a PGRN-associated disorder.

2. The method of claim 1, further comprising treating another test sample or subject with another compound and selecting a candidate compound that improves the one or more BMP species levels.

3. The method of claim 1, further comprising:
(d) maintaining or adjusting the amount or frequency of administration of the compound to the test sample or subject; and
(e) administering the compound to the test sample or to the subject.

4. A method for identifying a subject having, or at risk of having, a PGRN-associated disorder, the method comprising:
(a) measuring the abundance of one or more BMP species in a test sample from a subject;
(b) comparing the difference in abundance between the one or more BMP species measured in (a) and one or more reference values; and
(c) determining from the comparison whether the subject has a PGRN-associated disorder.

5. The method of claim 4, further comprising administering to the subject a compound for improving the one or more BMP species levels for treating a PGRN-associated disorder.

6. The method of any one of claim 1 to 3 or 5, wherein the compound is PGRN, a PGRN derivative, or pharmaceutical compositions thereof.

7. The method of any one of the preceding claims, wherein the reference value is measured in a reference sample obtained from a reference subject or a population of reference subjects.

8. The method of claim 7, wherein the reference subject or population of reference subjects is a healthy control.

9. The method of claim 7, wherein the reference subject or population of reference subjects do not have a PGRN-associated disorder or a decreased level of PGRN.

10. The method of any one of the preceding claims, wherein a subject having, or at risk of having, a PGRN-associated disorder has increased BMP species levels in bone marrow derived macrophages compared to a healthy control or a control not related to a PGRN-associated disorder.

11. The method of any one of the preceding claims, wherein a subject having, or at risk of having, a PGRN-associated disorder has decreased BMP species levels in liver, brain, cerebrospinal fluid, plasma, or urine compared to a healthy control or a control not related to a PGRN-associated disorder.

12. The method of any one of the preceding claims, wherein the abundance of a BMP species in the test sample of a subject having, or at risk of having, a PGRN-associated disorder has at least about a 1.2-fold, 1.5-fold, or 2-fold difference compared to a reference value of a control such as a healthy control or a control not related to a PGRN-associated disorder.

13. The method of any one of the preceding claims, wherein the abundance of a BMP species in the test sample of a subject having, or at risk of having, a PGRN-associated disorder has at least about a 1.2-fold to about 4-fold difference compared to a reference value of a control such as a healthy control or a control not related to a PGRN-associated disorder.

14. The method of any one of claims 1 to 7, wherein the reference value is the BMP species value prior to treatment.

15. The method of any one of the preceding claims, wherein the improved BMP species level is an improvement over the BMP species level prior to treatment relative to the reference value of a control such as a healthy control or a control not related to a PGRN-associated disorder.

16. The method of claim 15, wherein the improved BMP species level has a difference compared to the control of less than 15%, 10%, or 5%.

17. The method of any one of the preceding claims, wherein the test or reference sample or one or more reference values comprise or relate to a cell, a tissue, whole blood, plasma, serum, cerebrospinal fluid, interstitial fluid, sputum, urine, lymph, or a combination thereof.

18. The method of claim 17, wherein the cell is a peripheral blood mononuclear cell (PBMC), a bone marrow-derived macrophage (BMDM), a retinal pigmented epithelial (RPE) cell, a blood cell, an erythrocyte, a leukocyte, a neural cell, a microglial cell, a brain cell, a cerebral cortex cell, a spinal cord cell, a bone marrow cell, a liver cell, a kidney cell, a splenic cell, a lung cell, an eye cell, a chorionic villus cell, a muscle cell, a skin cell, a fibroblast, a heart cell, a lymph node cell, or a combination thereof.

19. The method of claim 17 or 18, wherein the cell is a cultured cell.

20. The method of claim 19, wherein the cultured cell is a BMDM or an RPE cell.

21. The method of claim 17, wherein the tissue comprises brain tissue, cerebral cortex tissue, spinal cord tissue, liver tissue, kidney tissue, muscle tissue, heart tissue, eye tissue, retinal tissue, a lymph node, bone marrow, skin tissue, blood vessel tissue, lung tissue, spleen tissue, valvular tissue, or a combination thereof.

22. The method of any one of the preceding claims, wherein the test sample comprises an endosome, a lysosome, an extracellular vesicle, an exosome, a microvesicle, or a combination thereof.

23. The method of any one of the preceding claims, wherein the one or more BMP species comprise two or more BMP species.

24. The method of any one of the preceding claims, wherein the one or more BMP species comprise BMP(16:0_18:1), BMP(16:0_18:2), BMP(18:0_18:0), BMP(18:0_18:1), BMP(18:1_18:1), BMP(16:0_20:3), BMP(18:1_20:2), BMP(18:0_20:4), BMP(16:0_22:5), BMP(20:4_20:4), BMP(22:6_22:6), BMP(20:4_20:5), BMP(18:2_18:2), BMP(16:0_20:4), BMP(18:0_18:2), BMP(18:0e_22:6), BMP(18:1e_20:4), BMP(20:4_22:6), BMP(18:0e_20:4), BMP(18:2_20:4), BMP(18:1_22:6), BMP(18:1_20:4), BMP(18:0_22:6), or a combination thereof.

25. The method of any one of the preceding claims, wherein the one or more BMP species comprise BMP(18:1_18:1), BMP(18:0_20:4), BMP(20:4_20:4), BMP(22:6_22:6), BMP(20:4_22:6), BMP(18:1_22:6), BMP(18:1_20:4), BMP(18:0_22:6), BMP(18:3_22:5), or a combination thereof.

26. The method of any one of the preceding claims, wherein the test sample comprises a BMDM and the one or more BMP species comprise BMP(18:1_18:1).

27. The method of any one of the preceding claims, wherein the test sample comprises plasma, urine, cerebrospinal fluid (CSF), and/or brain or liver tissue, and the one or more BMP species comprise BMP(22:6_22:6).

28. The method of any one of the preceding claims, wherein the test sample comprises liver tissue and the one or more BMP species comprise BMP(22:6_22:6), BMP(18:3_22:5), or a combination thereof.

29. The method of any one of the preceding claims, wherein the test sample comprises CSF or urine and the one or more BMP species comprise BMP(22:6_22:6).

30. The method of any one of the preceding claims, wherein the test sample comprises CSF and the one or more BMP species comprise BMP(18:1_18:1).

31. The method of any one of the preceding claims, wherein the test sample comprises microglia and the one or more BMP species comprise BMP(18:3_22:5).

32. The method of any one of the preceding claims, wherein the abundance of the one or more BMP species is measured using liquid chromatography-mass spectrometry (LC-MS), liquid chromatography-tandem mass spectrometry (LC-MS/MS), gas chromatography-mass spectrometry (GC-MS), gas chromatography-tandem mass spectrometry (GC-MS/MS), enzyme-linked immunosorbent assay (ELISA), or a combination thereof.

33. The method of any one of the preceding claims, wherein an internal BMP standard is used when measuring the abundance of the one or more BMP species.

34. The method of claim 33, wherein the internal BMP standard comprises a BMP species that is not naturally present in the subject and/or the reference subject or population of reference subjects.

35. The method of claim 33 or 34, wherein the internal BMP standard comprises BMP(14:0_14:0).

36. The method of any one of the preceding claims, wherein the PGRN-associated disorder is a disorder related to PGRN expression, processing, glycosylation, cellular uptake, trafficking, and/or function.

37. The method of any one of the preceding claims, wherein the subject has one or more mutations in granulin (GRN) gene.

38. The method of any one of the preceding claims, wherein the PGRN-associated disorder is associated with a decreased PGRN level.

39. The method of any one of the preceding claims, wherein the PGRN-associated disorder is a neurodegenerative disease.

40. The method of any one of the preceding claims, wherein the PGRN-associated disorder is a disease selected from the group consisting of frontotemporal dementia (FTD), neuronal ceroid lipofuscinosis (NCL), Niemann-Pick disease type A (NPA), Niemann-Pick disease type B (NPB), Niemann-Pick disease type C (NPC), C9ORF72-associated amyotrophic lateral sclerosis (ALS)/FTD, sporadic ALS, Alzheimer's disease (AD), atherosclerosis, Gaucher disease, Parkinson's disease, and age-related macular degeneration (AMD).

41. The method of any one of the preceding claims, wherein the subject and/or the reference subject is a human, a non-human primate, a rodent, a dog, or a pig.

42. The method of any one of the preceding claims, wherein the subject having a disorder associated with decreased PGRN levels is a PGRN knockout mouse or PGRN knockout rat.

43. A kit for testing a compound or a dosing regimen thereof for treating a PGRN-associated disorder, the kit comprising a BMP standard for measuring the abundance of one or more BMP species in a test sample from the subject.

44. The kit of claim 43, wherein the BMP standard comprises a BMP species that is not naturally present in the subject.

45. The kit of claim 43 or 44, wherein the BMP standard comprises BMP(14:0_14:0).

46. The kit of any one of claims 43 to 45, wherein the kit further comprises reagents for obtaining the sample from the subject, processing the sample, measuring the abundance of the one or more BMP species, or a combination thereof.

47. The kit of any one of claims 43 to 46, wherein the kit further comprises instructions for use.


SEQUENCE LISTING (2 sequences)

SEQ ID NO: 1 - protein, 10 aa, synthetic construct
  REGION 1..10: note = Description of Artificial Sequence: Synthetic peptide
GGGGSGGGGS

SEQ ID NO: 2 - protein, 5 aa, synthetic construct
  REGION 1..5: note = Description of Artificial Sequence: Synthetic peptide
GGGGS